(19) United States
(12) Patent Application Publication
(10) Pub. No.: US 2022/0051582 A1
(43) Pub. Date: Feb. 17, 2022

(54) SYSTEM AND METHOD FOR MINDSET TRAINING

(71) Applicant: Thomas Sy, Huntington Beach, CA (US)

(72) Inventor: Thomas Sy, Huntington Beach, CA (US)

(21) Appl. No.: 16/993,895

(22) Filed: Aug. 14, 2020

(51) Int. Cl. G09B 5/12; H04L 12/58; G09B 19/00

(52) U.S. Cl. CPC G09B 5/12; A63F 13/80; G09B 19/00; H04L 51/32

(57) ABSTRACT

The present invention relates to a software-based system and method of mindset training with gamification, implemented on stand-alone computing devices or over a network using computing devices. More specifically, the present invention relates to modifying the mindset of users of the system and method by means of mindset goal specification and corresponding mindset content curation, mindset conditioning games, social network engagement, and performance tracking.

### FIELD

The present invention generally relates to a system and method for training the mindset of a subject, and more particularly, to a system and method for mindset training applications for mobile users.

### BACKGROUND

A number of methods have been developed to assess individuals' associations, with the Implicit Association Test (IAT) being the most popular. The IAT was developed and disclosed in U.S. Pat. No. 10,217,526 issued to Greenwald et al. (hereinafter “Greenwald”) Similar methods have been disclosed for assessing individuals' associations, as discussed in U.S. Patent Publication No. 2012/0021399 by Cvencek et al., U.S. Patent Publication No. 2018/0308383 by Draine, U.S. Patent Publication No. 2018/0293599 by Reid et al., and U.S. Pat. No. 10,043,411 issued to Ohme et al. The focus of these assessment measures is to provide individuals with an understanding of themselves. For example, the IAT provides an assessment of individuals' implicit biases (e.g., racial, gender, religion, age, etc.). The assessment measures do not change associations but can be utilized to educate individuals by providing insights about their automatic reactions (System 1 reflexive thinking).

A few technology based methods for changing associations have been disclosed. U.S. Patent Publication No. 2005/0250082 by Baldwin et al. (hereinafter “Baldwin”), and the associated website www.mindhabits.com, disclose a method for improving self-esteem via Associative Conditioning. Users are tasked to locate their names hidden within an array of distractor stimuli. Upon selecting their names, users are presented with a positive stimulus (e.g., a photo of a smiling face). Over multiple repetitive trials, users come to associate their names or themselves with the positive stimulus, and through this association improve self-esteem.

Baldwin also disclosed a deconditioning method. Deconditioning involves a process where previously learned associations are unlearned by changing attentional focus. Typically, attentional focus training challenges users to suppress responses to stimuli to which they are prone toward an unhealthy psychological response, such as depression and anxiety. For example, individuals with depression are more prone to focus and linger on glum content, which can be the cause of their depression. Accordingly, deconditioning procedures teach users to suppress their response to glum stimuli. A common deconditioning procedure for depression involves challenging users to locate a photo of a generic grinning face hidden within an array of distractor photos of generic sad faces. This procedure reduces depression by teaching users to suppress negative stimuli (generic sad faces) that cause depression. A limitation with Associative Conditioning and Deconditioning methods disclosed in prior art, including those cited herein, concern whether generic stimulus content validly represent the mindset goal of interest for a specific user, as will be discussed later. Moreover, the disclosed variants of the method (e.g., varying the pairing of stimuli, the strength of the stimuli reinforcement, users' awareness of the procedures, etc.) lack empirical support.

Patent application WO2010/100208 by Delany et al. (hereinafter “Delany”) teaches a variant of the deconditioning method involving the N-back task. Delany differentiates their method from others, such as Baldwin, by claiming that the N-Back variant of the deconditioning method can improve cognitive function (i.e., IQ), although this is a source of much debate among scientists.

U.S. Patent Publication No. 2008/0261186 by Hurling (hereinafter “Hurling”) disclosed a method to develop and/or alter implicit attitudes towards objects by associating those objects to users. The users serve as the unconditioned stimulus (i.e., the emotional content for which the objects becomes associated) and assumes that users have positive self-evaluations such that the objects will come to be judged positively via association. This is untenable given that individuals vary significantly in self-evaluation. Indeed, this is evident in Baldwin where the intervention is designed to improve self-esteem, recognizing that individuals may not always have positive self-evaluations. Based on the classic approach-avoidance paradigm, Hurling's method also incorporates a behavioral component whereby objects are positively associated with the individual by “pulling” the object towards the individual and dissociated from the individual by “pushing” the object away from the individual.

U.S. Patent Publication No. 2015/0086952 by Tsoneva et al. (hereinafter “Tsoneva”) disclosed a method of Associative Conditioning by forming associations with a characteristic feature that is common to both stimuli (e.g., a green apple and a pet playing in a grassy field; both possess the color green). Tsoneva emphasizes a passive user method where the device (e.g., a computer system, and not the user) determines the common characteristic feature and applies the psychological technique of “priming” the user with the stimuli (i.e., subliminal or subtle presentation of the stimuli that is outside of the individual's conscious awareness). Similar to Hurling, the user serves as the unconditioned stimulus, with the difference being whether the user serves as the unconditioned stimulus directly or indirectly (e.g., via the family of the user). In both instances, direct and indirect aspects of the user serve as emotional content that elicit an evaluative response (positive or negative).

Two peer reviewed scientific studies disclosed similar methods of applying a mobile application of Associative Conditioning. Users in the first study (a clinical population) played a mobile game that induced a negative association for self-injury (conditioned stimulus) by pairing it with photos of snakes, spiders, and other insects (unconditioned stimulus). Consequently, users engaged in less self-cutting behavior after the intervention, in comparison to a control group that did not receive the intervention. Inspired by the first study, the second study designed a similar intervention, although his study induced a positive association of body image by pairing photos of users with photos of people smiling). In both studies, it is not clear whether the conditioned stimuli, i.e., self-injury or body image, were validly represented by generic photos of cutting-behaviors and photos of users. These photos can represent many things and different things for different users. For example, the photos of users in the second study could be a representation of users' body image or their personality, age, gender, race or whatever is most salient and relevant to the users. The issue holds true for all of the cited patents herein but is highlighted here because scientific studies are held to a higher standard and the hallmark of robust experimental studies requires a “manipulation check” to ensure that the variables of interest are validly represented.

Prior Art Disadvantages

The various methods disclosed in prior art have questionable validity and/or are inconsistent with scientific findings including: varying the order or sequence of stimuli (e.g., presenting unconditioned stimulus first followed by the conditioned stimulus, and vice versa), the requirement for stimuli to be subliminal (outside the awareness of individuals), the requirement for stimuli to have common incidental features of no salient conceptual significance, reliance on individuals as the basis for the US or emotional content (individuals vary significantly on self-evaluation, e.g., self-esteem), and varying the reinforcement strength of the US (intensity of the emotional content).

In addition, prior art suffers from one or more of the following disadvantages:

A. Professional-Centric Method. Users are dependent on skilled professionals (e.g., scientists, researchers, clinicians, application developers, etc.) to develop mindset training interventions. This leaves many users without intervention programs to address their specific mindset goals because skilled professionals have focused only on a limited range of mindset domains. Given the plethora of potential mindset domains requiring interventions, there are a lot of mindset domains with no existing intervention available.

B. Generic Content. Universal and generic content (e.g., stock photos of generic individuals with no relation to users) that are curated by skilled professionals 1) require considerable time to curate, test, and validate, and 2) lacks or has less relevance for users, resulting in less engagement for users and less efficacy in mindset training. Moreover, there is no method or system for representing individual users' unique mindset. Although the prior art (e.g., Baldwin, Hurling, and Tsoneva) has disclosed methods that included a personal features of users (i.e., name, photo of users or their family), the personal feature is only a representation of users (i.e., a user's individual identity as a person) and not a representation of users' unique mindsets (i.e., users' unique psychological orientations and predispositions).

C. Ambiguous Representation of Content. Users have Unique Mindsets. Generic content curated by skilled professionals do not necessary represent the same mindset across different users. For example, Tsoneva disclosed in their method an example of representing “healthy lifestyle” with an image of a swimmer in water (conditioned stimulus) that can be paired with a positive image (smiling face) to improve users' liking for exercising. However, for users with aquaphobia (fear of water), the image is most likely to serve as a representation of fear. Alternatively, the image of a swimmer may serve as a negative unconditioned stimulus (instead of conditioned stimulus) if users dislike swimming, being in cold water, or physical exertion. This imprecision of generic content representation hinders the efficacy of mindset training.

D. Static Content. Once stimulus content is curated by skilled professionals, they remain static. New content is not curated in a continuous basis such that the same content is used throughout training. Static content loses its representation for a given mindset domain overtime (e.g., a photo of a dog that initially represents and elicits happiness fails to do so over time as users become habituated to the same photo). Moreover, users' motivation for training declines overtime as users become habituated to static content.

E. Static and Rigid System and Method. Existing systems and methods lack flexibility and customization, comprising of interventions limited to conditioning a single mindset with a single game or task.

F. Narrow Application of Dual Thinking System. Mindsets are formed and changed based on a dual thinking system, namely System 1 (reflexive thinking) and System 2 (reflective thinking). Prior art's reliance on Associative Conditioning methods comprising solely of System 1 processes hampers the effectiveness of training.

G. Limited User Engagement. Intervention efficacy is dampened due to poor user engagement given a lack of or limited engagement features. Prior art either does not have any gamification or incentive features, or possess only a single feature.

H. Dual Stimuli Associative Conditioning. Associative Conditioning methods of prior art requires more than one stimuli (conditioned stimulus and unconditioned stimulus). The dual stimuli requirement is inefficient and creates additional complexity that affect training efficacy.

### SUMMARY

Prior art does not teach or suggest an effective system and method for mindset training with gamification, comprising applications that can be implemented on mobile devices and software applications that can be implemented on one or more server computers that are configured to 1) permit users to be in charge of their own mindset training interventions, 2) permit users to represent their unique mindset, involving motivations, attitudes, states, and traits, via multiple modalities including, visual, auditory, gustatory, olfactory, and tactile, or a combination thereof, 3) permit users to specify and input one or more mindset training goals, 4) permit users to curate mindset content that is germane and specific to users, 5) permit users to curate user-unique content that simultaneously represents a conditioned stimulus and a unconditioned stimulus (e.g., a mindset domain and its associated positive or negative characteristics), 6) permits users to train their mindset goals via the curation of mindset content, 7) transform user-unique content into game stimuli by the computer for use as inputs in conditioning games, 8) permit users to train their mindset goals by playing conditioning games, 9) provide social network functionalities for user engagement, motivation, and support, and 9) permit users to track performance and review progress for their mindset training.

Accordingly, several advantages of one or more aspects include a user-centric mindset training system and method that 1) permit users to develop their own interventions by specifying their own mindset goals and curating the corresponding mindset content; 2) solve the previously unidentified problem of using generic content in Associative Conditioning by providing means for curating user-unique content; 3) provide means for precisely representing users' unique mindset with visual, auditory, gustatory, olfactory, and tactile content, or a combination thereof—thus increases the precision of mindset representation and consequently, the efficacy of training; 4) provide means for curating dynamic mindset content that strengthens users' connection with the content—the strong connection with novel and salient content results in higher user engagement and training efficacy; 5) provide a single flexible and customizable intervention that can accommodate one or multiple mindset goals for training, as well as providing multiple conditioning games; 6) produce synergistic training effects by integrating System 1 and System 2 processes in a single intervention that enhances training efficacy; 7) increase user engagement via gamification involving multiple game activities (e.g., content curation, conditioning games, etc.), performance review metrics, and social network engagement; and 8) permit users to curate content that simultaneously represents a conditioned stimulus and a unconditioned stimulus (e.g., a mindset domain and its associated positive or negative characteristics), and thereby reduce complexity while improving efficiency that result in higher training efficacy. These and other benefits of one or more aspects will become apparent from a consideration of the ensuing description and accompanying drawings.

### BRIEF DESCRIPTION OF THE DRAWINGS

FIG. 1A is a simplified block diagram that illustrates an example communications system into which an exemplary embodiment of the disclosure may be implemented.

FIG. 1B is a simplified block diagram that illustrates an example implementation of an example device, in accordance with an exemplary embodiment of the disclosure.

FIG. 2 is a simplified block diagram that illustrates an example of functional blocks of a system implemented, in accordance with an exemplary embodiment of the disclosure.

FIGS. 3A to 3E are simplified flow diagrams illustrating example system level process operations, in accordance with an exemplary embodiment of the disclosure.

FIG. 4 shows a screen view of an example sign-up screen, in accordance with an exemplary embodiment of the disclosure.

FIGS. 5A to 5E show screen views of an example process for creating a user profile by uploading a photo, in accordance with an exemplary embodiment of the disclosure.

FIGS. 6A to 6E show screen views of an example introduction screen providing an overview of the functionality of the application to users, in accordance with an exemplary embodiment of the disclosure.

FIG. 7 shows a screen view of an example user profile screen, in accordance with an exemplary embodiment of the disclosure.

FIG. 8A shows a screen view of an example set of instructions for guiding users in the identification and representation of mindset goals, in accordance with an exemplary embodiment of the disclosure.

FIG. 8B shows a screen view of an example mindset curation graphical user interface, along with a set of instructions describing means for curating and uploading content representing mindset goals, in accordance with an exemplary embodiment of the disclosure.

FIGS. 8C to 8L show screen views of example processes for curating and uploading content representing mindset goals, in accordance with an exemplary embodiment of the disclosure.

FIGS. 9A and 9B show screen views of an example social network Graphic User Interface, in accordance with an exemplary embodiment of the disclosure.

FIG. 10 shows a screen view of an example profile screen of a member from a user's social network, along with the member's mindset content, in accordance with an exemplary embodiment of the disclosure.

FIG. 11 shows a screen view of an example profile screen for a mindset content item, in accordance with an exemplary embodiment of the disclosure.

FIGS. 12A and 12B show screen views of an example conditioning games graphical user interface, in accordance with an exemplary embodiment of the disclosure.

FIG. 13 shows a sequence of screen views of an example set of instructions for a conditioning game, in accordance with an exemplary embodiment of the disclosure.

FIGS. 14 and 15 show a sequence of screen views of examples of conditioning games presenting mindset content stimuli during a conditioning session, in accordance with an exemplary embodiment of the disclosure.

FIG. 16 shows a screen view of an example query screen for a conditioning game, in accordance with an exemplary embodiment of the disclosure.

FIGS. 17A to 17D show screen views of example feedback for conditioning games provided to users during a conditioning session, in accordance with an exemplary embodiment of the disclosure.

FIGS. 18A to 18C show screen views of an example performance review graphical user interface, in accordance with an exemplary embodiment of the disclosure.

FIG. 19 is a chart in accordance with an exemplary embodiment of the disclosure.

### DETAILED DESCRIPTION

Self-improvement is a perennial goal in all aspects of life, both personally and professionally. For example, individuals want to improve their personal happiness, as well as become more effective contributors and leaders in society. Accordingly, various training interventions have been developed, comprising of methods for improving knowledge, skills, and mindsets.

Prevailing methods center on providing knowledge and behavioral skills, with little or no consideration of the corresponding need to train and align individuals' mindset. The essence of knowledge-based methods is to provide information related to understanding oneself (e.g., insights on the sources of personal happiness or characteristics of effective leaders). The essence of skill-based methods are to train key behaviors to manifest one's self-improvement goals (e.g., writing a letter of appreciation to improve happiness or using body language to appear more like a charismatic leader). However, knowledge- and skill-based methods alone may not be sufficient if individuals' mindset are not aligned. Despite having the requisite knowledge or skills, individuals will not realize their self-improvement goals until they are in the right state of mind.

If mindset is equally important as knowledge and skills, then what explains the paucity of interventions for mindset training? Self-improvement interventions frequently are limited to teaching knowledge and skills because they are easier to develop given the readily available methods. For example, we can train knowledge via lectures, presentations, and books, and train skills via demonstrations, role-play, and guided practice. In contrast, there are few known methods that can directly train individuals' mindset because of its latent nature. In comparison to the concreteness of information and behavior that can be observed (physically manifested) and manipulated directly, mindsets are intrapsychic phenomena residing in the minds of individuals. That is, mindsets are latent constructs that can never be directly observed. Because mindsets are abstract and can never be directly observed, it poses a difficult challenge for developing training interventions. Consequently, there is a need for systems and methods that provide means for training individuals' mindset.

Mindset

Mindsets are wide encompassing phenomena. A search in the science literature with the term “mindset” yields a host of domains, such as “global,” “entrepreneurial,” “success,” “fixed,” “growth,” “courageous,” “ethical,” “innovative,” “millennial,” “design thinking,” “consumer,” “follower,” “happiness,” “leadership,” and among others. Although wide-ranging, mindsets can be summarized as a psychological orientation or predisposition, including motivations (e.g. desire to exercise more), attitudes (e.g., implicit biases), states (e.g., happiness), and traits (e.g., charismatic leader).

Dual Thinking Systems

Research indicates that mindsets are formed and changed based on a dual thinking system that govern human behavior. The dual thinking system has wide support, counting over twelve different theoretical accounts, with some prominent ones being propositional versus associative learning, rule-based versus associative learning, and explicit versus implicit learning. These accounts can be subsumed under an overarching System 1 versus System 2 model. System 1 is reflexive thinking that is fast and can operate in automatic (involuntary) and subconsciously fashion. System 2 is reflective thinking that is slow and operate in controlled (deliberate) and conscious fashion. Examples of System 1 reflexive thinking include automatic reactions to threats (e.g., jumping out of the pathway of a speeding car) or implicit biases (e.g., racial, gender, age, etc.) that operate outside of conscious awareness. Examples of System 2 reflective thinking include solving a challenging math problem or writing a bestselling novel that require conscious and deliberate effort. System 1 is always active and thus the default thinking system. System 2 typically is engaged when individuals have sufficient motivation and opportunity.

Although System 1 and System 2 interact and have mutual influence, they are often characterized as separate and opposing systems. Consequently, scientists typically study the two systems separately. Similarly, the development of interventions programs also fall along this bifurcated line, with the majority designed on the tenets of System 2. For example, as discussed in U.S. Patent Publication No. 2018/0068573A1 by Costa et al., the majority of training interventions involving computing technology are developed on the basis of System 2 (94%, 165 interventions) versus System 1 (6%, 11 interventions). Notably, none addressed mindset training. Paralleling the above discussion, prevailing training methods that target knowledge and skill development are created on the basis of System 2. The acquisition of knowledge and skills via the methods of lectures, presentations, books, demonstrations, role-play, guided practice, and among others require considerable conscious and deliberate effort (hallmark characteristics of System 2). Contrasting interventions targeting mindset development are created on the basis of System 1, notably Pavlovian or Associative Conditioning.

Associative Conditioning

Associative Conditioning reflects System 1 processes and is defined as a change in behavior that is due to the pairing of stimuli, and one subvariant is evaluative conditioning, which is similarly defined as a change in liking that is due to the pairing of stimuli. Associative and evaluative conditioning are comprised of similar procedures, with the former involving changes in observable response and the latter involving changes in (evaluative) liking. Another subvariant is attribute conditioning, which is defined as a change in attribute assessment (e.g., a person's characteristic) due to the pairing of stimuli. As they reflect the same procedure, Associative Conditioning will be referenced herein. Associative Conditioning reflects a change in orientation of a first neutral stimulus (the outcome) cause by its repeated pairing with a second (positive or negative) stimulus (the procedure). The first stimulus is the conditioned stimulus (CS) and the second stimulus is the unconditioned stimulus (US). In a typical Associative Conditioning procedure, stimuli are typically selected for their symbolic representation for a construct of interest. For example, a stimulus photo of individuals swimming or biking can symbolically represent exercising activities, and a stimulus photo of smiling faces can symbolically represent positivity or happiness. Stimuli can take the form of various modalities, including visual (e.g., photos), auditory (e.g., sounds), gustatory (e.g., taste), olfactory (e.g., smell), and tactile (e.g., vibration). A CS is evaluated more positively when it is paired with a positive US and more negatively when it is paired with a negative US. For example, individuals' attitudes towards exercising became more positive when photos of exercisers (e.g., swimming and biking; CS) were repeatedly paired with photos of smiling faces (US) that unconditionally triggers a positive evaluation. Similarly, attitudes about self-cutting behavior for a clinical population became more negative when photos of self-cutting behavior (CS) were repeatedly paired with photos of snakes, spiders, and other insects (US) that unconditionally trigger a negative evaluation.

Associative Conditioning has strong validity with hundreds of studies dating back more than a century. It is a robust and widely studied effect demonstrating broad implications. It has wide applications in a variety of areas (e.g., learning psychology, social psychology, advertising, creativity, emotion, neuroscience, nutrition, clinical psychology, exercise, etc.) and populations (e.g., children, adults, men, women, different racial backgrounds, nonclinical and clinical samples, etc.). Moreover, the procedures of Associative Conditioning works with a variety of modalities, including visual (photos), auditory (sounds), gustatory (taste), olfactory (smell), and tactile (vibration). Despite its long history and prolific scientific studies, there are relatively few practical applications.

A system and method for mindset training for mobile users is disclosed in accordance with exemplary embodiments. The exemplary embodiments disclose system components and associated programming platforms that provide application programming interfaces for supporting a system and method of mindset training applications. The application programming interfaces support the features and functionalities, access to databases, and the data and information flow for web or mobile based device applications that receive information associated with users of the applications or deliver interventions to users of the applications. The interventions may be in the form of game activities (e.g., curating mindset content, playing games, or engaging with social network), notifications (e.g., visual, audible or haptic), instructions (e.g., directives to perform a certain action) or other data information delivery to users.

In one exemplary embodiment, the system and method could be implemented as a mindset training system comprising applications that may be implemented on mobile devices and software applications that may be implemented on one or more server computers. The mobile devices may be smart phones or tablets, or other types of mobile devices, such as wearable devices (e.g., smartwatch, smartglass, virtual reality headset, etc.) that include sensors for detecting activity or sensing data on physical or environmental conditions. The exemplary embodiment may also include web based applications accessible using desktop or laptop computers. The exemplary embodiment of the mindset training system may also include functional modules implemented on one or more server computers that control data collection and integration, data analysis, and interventions with the users.

In the exemplary embodiment, the mobile devices could be utilized to receive, through user input or sensors, data and information associated with the users of the devices and transfer the data to the server computers. A mindset curation module (for mindset content) collects and organizes the data from users into various databases, for example and without limitation, organizing photos curated by users via a mobile device into various mindset goal categories, such as happiness, leadership, self-esteem, and so forth. The data could serve as input to a social network module and conditioning games module. A social network module could process the user level data stored in the various databases and constructs social networks. Various social networks of users may be formed based on the mindset goals (e.g., a social network comprising of users training for a happiness mindset goal).

A conditioning games module could process the user level data (e.g., mindset content) stored in the various databases and transform the data into one or more game stimuli for use as inputs in one or more conditioning games. An exemplary conditioning game could be a game wherein users recall randomized sequences of happiness photos that are stored in the various databases. The exemplary embodiment may also include a performance review module that tracks and measures users' performance and engagement with the mindset training system. For example and without limitation, the performance review module could analyze data from the various databases to provide users with performance feedback, such as scores on the game activities, performance ranking in comparison to others within the social network, time spent on the mindset training system, and so forth—in order to improve users' experience and engagement with the game and training efficacy.

### Exemplary Embodiment of Communication System

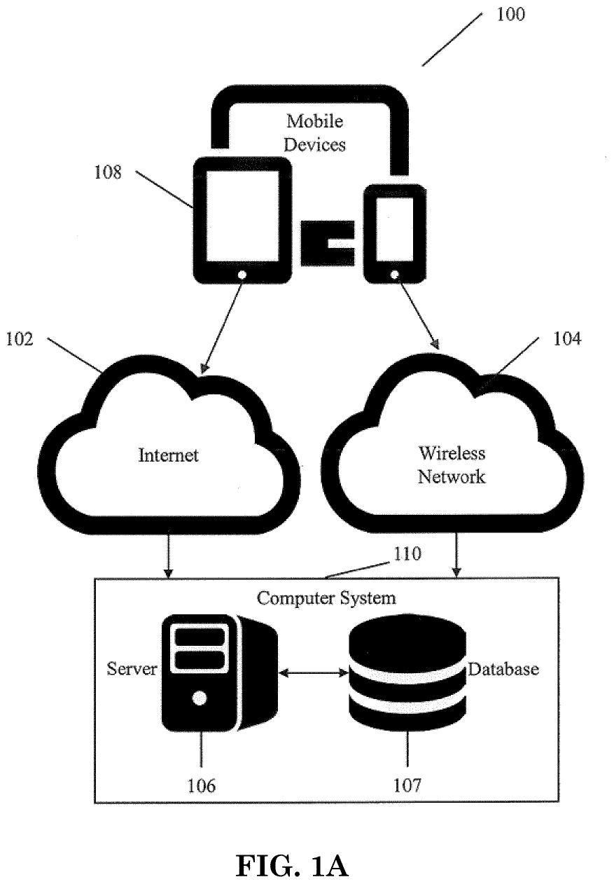

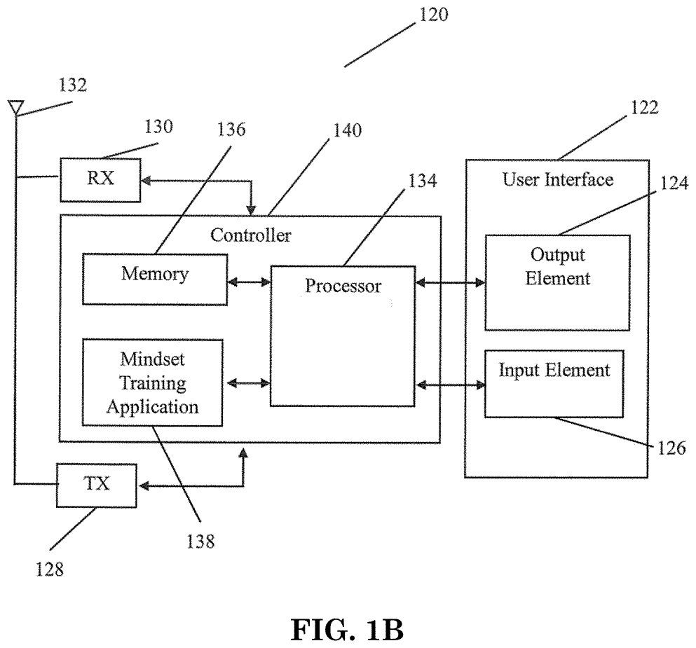

FIG. 1A is a simplified block diagram that illustrates an exemplary communication system into which an exemplary embodiment of the disclosure may be implemented. The communication system, shown generally by reference numeral 100, comprises mobile devices 108 which may be implemented, for example and without limitations, by smart phone, tablet, and laptop. Applications are implemented on the mobile devices 108 that provide the necessary data collection and training intervention functions of the embodiment. Any other type of device, such as a desktop personal computer, that may be operable to communicate the appropriate data may also be used in the system and method of the embodiment. Communication system 100 also includes internet network 102 and wireless network 104 over which the mobile devices 108 communicate with computer system 110 that includes a server 106 in communication with a database 107.

Internet network 102 and wireless network 104 may each comprise any appropriate radio communication and/or infrastructure equipment that implements communications between mobile devices 108 and server 106 using appropriate internet or wireless network communication protocols. The communications interfaces between mobile devices 108 and internet network 102 and wireless network 104, and, the interfaces between internet network 102, wireless network 104 and server 106, may also be implemented using any appropriate radio communication and/or infrastructure equipment. Although one server 106 and database 107 are represented in FIG. 1A, the functions represented by server 106 and database 107 may actually be implemented in one or more servers and one or more databases, and those functions or portions of those functions may be co-located or separated geographically in location. Server 106 may comprise software, hardware and software applications as necessary to implement the features and functions of the embodiment of the system and method.

### Exemplary Embodiment of Mobile Device

FIG. 1B is a simplified block diagram that illustrates an exemplary implementation of an exemplary mobile device in accordance with an exemplary embodiment of the disclosure. Mobile device 120 comprises antennae 132, receiver RX 130, transmitter TX 128, controller 140 and User Interface 122. RX 130 and TX 128 receive and transmit data and communications to and from server 106 over network 100. Controller 140 comprises processor 134, memory 136 and mindset training application 138, which, for example and without limitations, may be stored as program instructions in a memory of Controller 140.

In the exemplary embodiment, mindset training application 138 include mindset training application that cause mobile device 120 to function to communicate with server 106, collect and receive data, and, receive and present data at User Interface 122, as necessary to implement the functions of the embodiment. User Interface 122 comprises output element 124 for outputting data, notifications and training interventions to users, and input element 126 for receiving input from users. Output element 124 may comprise any interface for providing notifications and training interventions to users in a sensory manner, such as visual, audio, vibrating, or any other sensory means, including, for example and without limitations, a visual display, an alarm, an audio player, or a vibrator.

Input element 126 may comprise any element for inputting data to mobile device 120 such as, for example and without limitations, a keyboard, touch screen, audio input, motion detector, location detector, biometric sensor, or other type of sensor. In another embodiment, mobile device 120 may operate stand-alone software stored as program instructions in a memory of Controller 140. In this exemplary embodiment, mobile device 120 comprises of memory 136 for storing information, preferably permanently, which includes such data as required for operation of the mindset training application.

### Exemplary Embodiment of Functional Software Modules

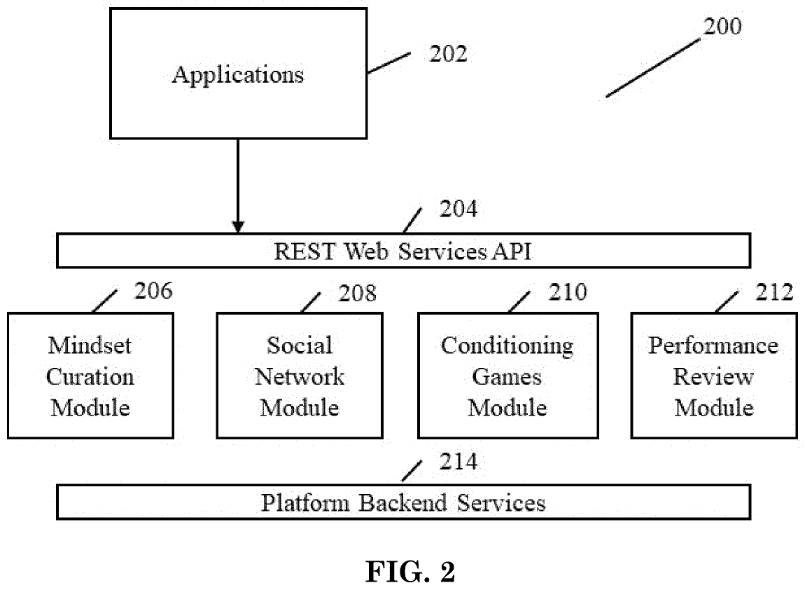

FIG. 2 is a simplified block diagram illustrating functional blocks of a system 200 implemented in accordance with an exemplary embodiment of the disclosure. FIG. 2 shows the functional software module blocks of mobile Applications 202, REST Web Services API 204, and Platform Backend Services 214 that comprise mindset curation module 206, social network module 208, conditioning games module 210, and performance review module 212. These functional modules may be implemented into a system, for example system 100 of FIG. 1A to create a system and method according to an exemplary embodiment of the disclosure.

Applications of module 202 may be implemented on the mobile devices 108 of FIG. 1A. Applications 202 may receive and collect data from users and the environment and deliver the training interventions to achieve the desired mindset. Applications 202 may be downloaded to the mobile device or be preinstalled. Applications 202 may include applications that interact directly with users through an interface, such as User Interface 122 of mobile device 120 of FIG. 1B, to receive inputs and provide outputs to users. Applications 202 may also include components such as, for example and without limitations, a Web-based application that can be accessed using a desktop computer or laptop with relatively more screen space, than a smart phone, that will allow the display of multiple forms of information simultaneously. Applications 202 may collect, receive, and deliver data in any form. For example and without limitation, Applications 202, interacting with the User interface 122 of mobile device 120 of FIG. 1B, may provide means for curating content by users such as photos videos, sounds, and so forth; interacting with other users on the social network module 208 such as liking and curating the content of others in a social network; play conditioning games; and reviewing performance history.

Applications 202 may also provide digital entry forms, e.g., survey forms, for the end-user to fill-in, or user interaction features such as page/screen views, with clicks or taps to enter responses or information. Applications 202 may also collect data such as location data from a GPS device or IP Addresses from web browsers. Collected data may also include data collected from sensors, for example and without limitations, microphones, accelerometers, thermometers, etc. These sensors may provide data on sound, positional location, movement, physical condition of mobile users, or the environment of a mobile device or of users of a mobile device. Applications 202 also may utilize the hardware aspects of mobile devices 108 to deliver interventions to users in order to implement the embodiments of the disclosure.

The Platform Backend Services 214 that comprise mindset curation module 206, social network module 208, conditioning games module 210, and performance review module 212 may be implemented in one or more servers such as server 106, and one or more databases such as database 107 of system 100 of FIG. 1A. Communication between the Applications 202 and Platform Backend Services may be implemented using REST Web Services API 204 as the interface. Various functions necessary for the Platform Backend Services 214 may be implemented by software or hardware or by a combination of software and hardware. Although the functions represented by mindset curation module 206, social network module 208, conditioning games module 210, and performance review module 212 are shown as single modules, those functions may actually be implemented in one or more servers and one or more databases, and those functions or portions of those functions may be co-located or separated geographically in location.

### Exemplary Embodiment of System Level Process Operation

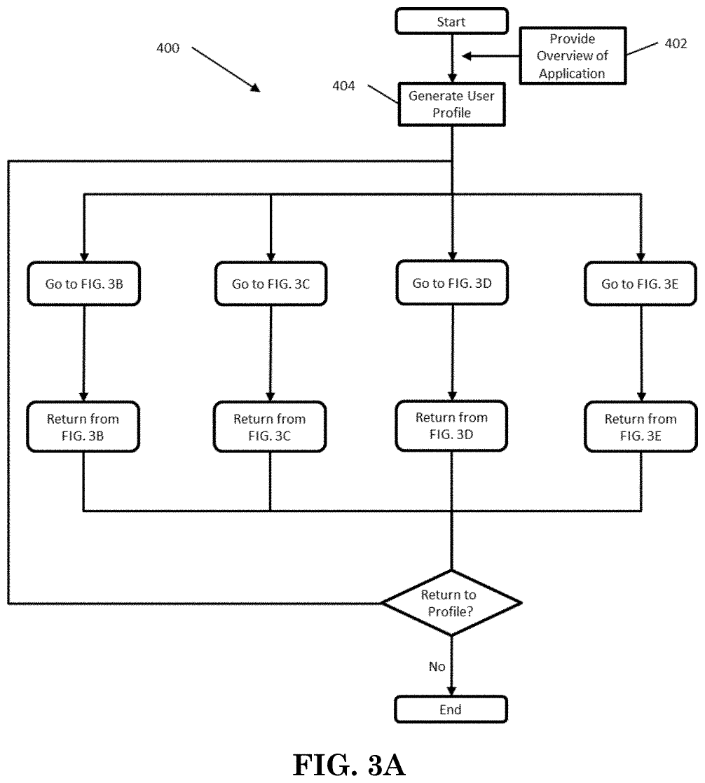

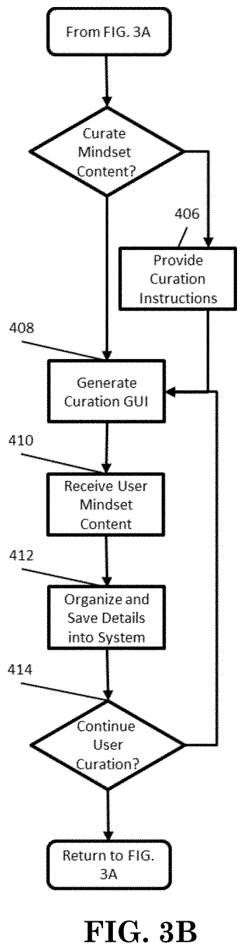

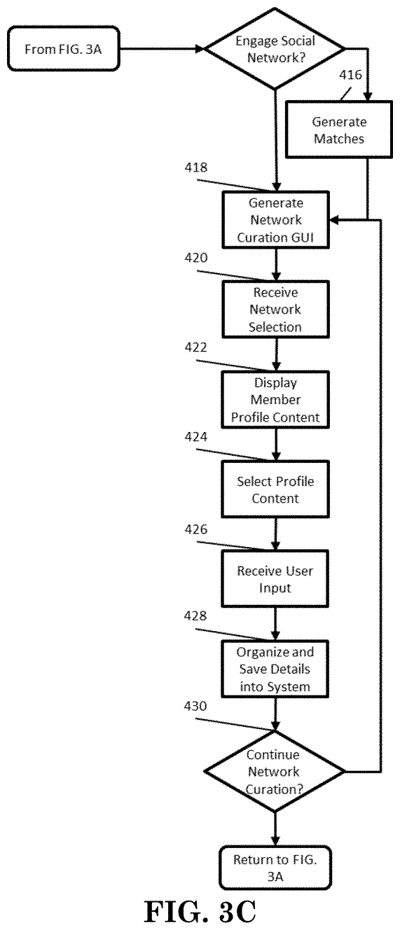

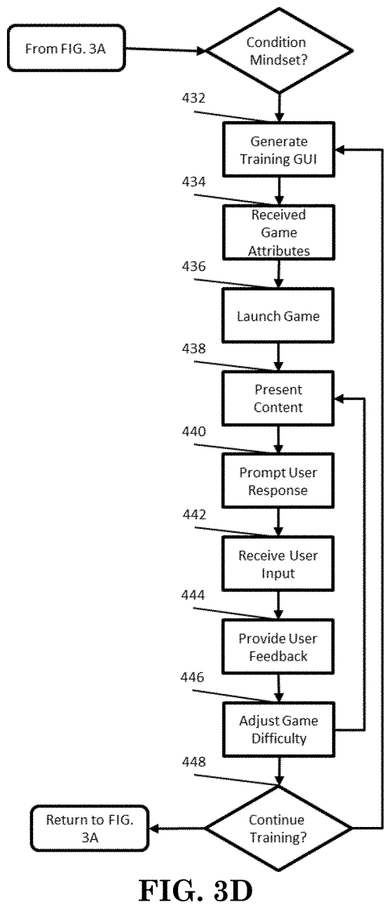

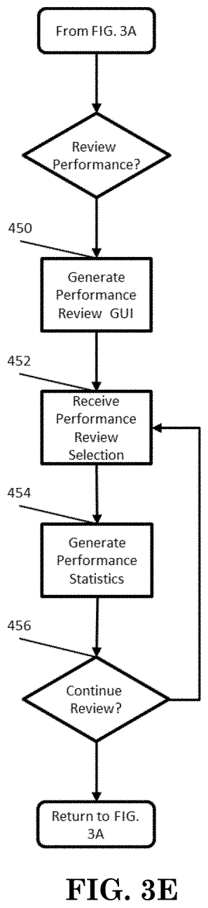

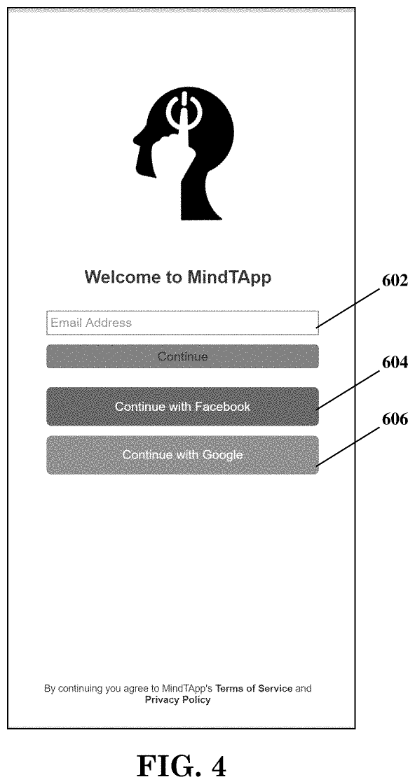

FIG. 3A is a simplified flow diagram 400 illustrating example system level process operations in accordance with an exemplary embodiment of the disclosure. FIGS. 3B to 3E are simplified flow diagrams of various branches of the flow diagram 400 (shown in FIG. 3A) illustrating example system level process operations in accordance with an exemplary embodiment. Users can use their mobile device 108 to access the computer system 110 sign-up page over the internet 102 or wireless network 104. FIG. 4 illustrates a screen view of one exemplary embodiment of the sign-up process. As shown in FIG. 4, users may sign-up via email 602 or using their existing Facebook 604 or Google accounts 606.

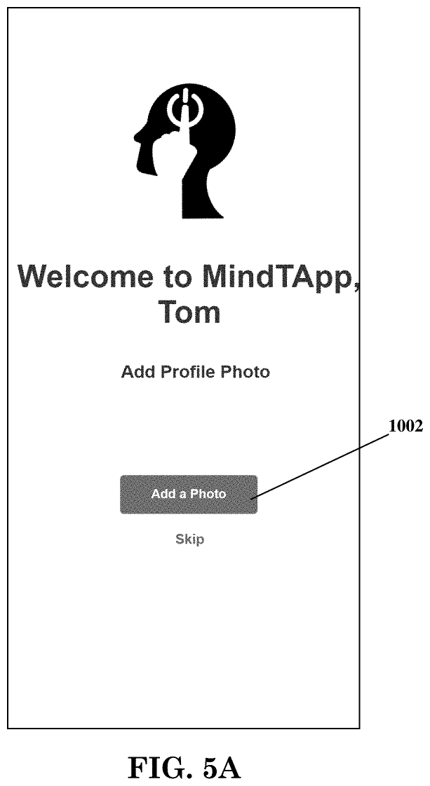

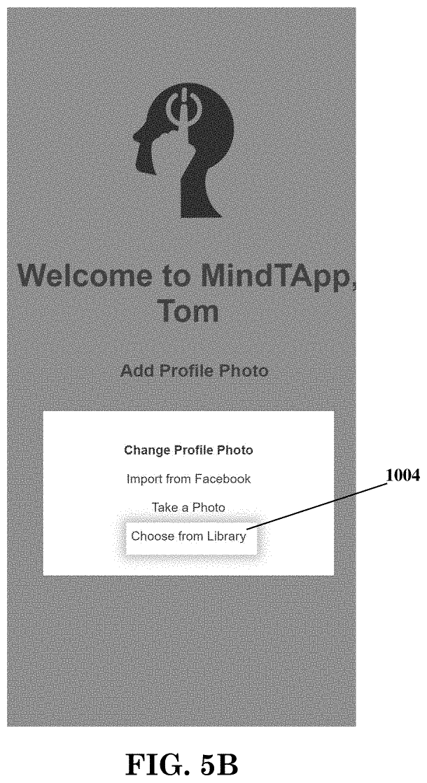

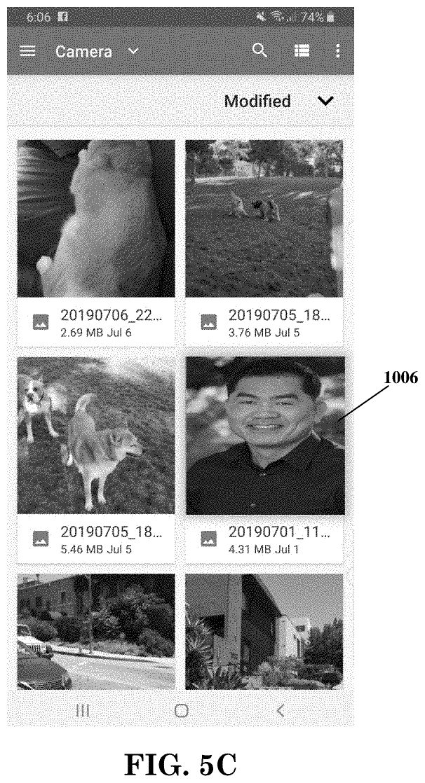

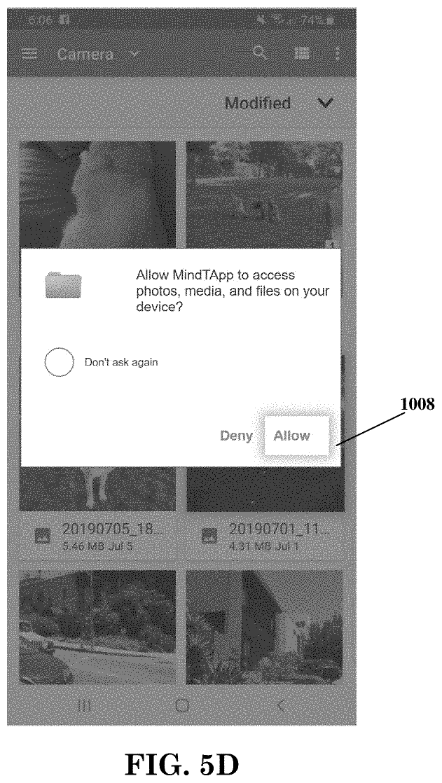

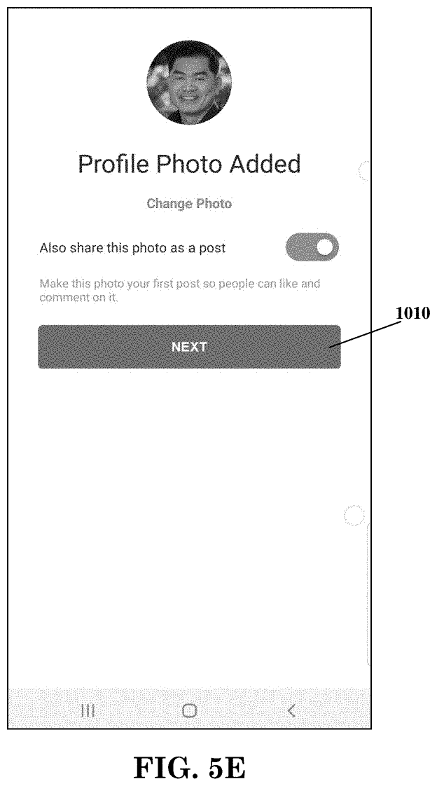

On receipt of an approved username and password, or successful login with Facebook or Google, the computer system 110 prompts users to create a user profile by uploading a photo. FIGS. 5A to 5E illustrate screen views of one exemplary embodiment of the process for creating a user profile photo. FIG. 5A illustrates one exemplary embodiment of a profile photo creation screen. As shown, the user is prompted to add a profile photo by selecting the “add a photo” button 1002. FIG. 5B illustrates a screen view of one exemplary embodiment showing various photo upload options, such as from Facebook, the photo library of the mobile device 108, or taking a self-photo using the camera function of the user interface 122 of the mobile device 108. In this example, the user chose to upload the photo from the photo library of the mobile device 108 by selecting “choose from library” button 1004. FIG. 5C illustrates one exemplary embodiment of a photo library. As shown the user makes a selection by selecting the photo 1006. FIG. 5D illustrates one exemplary embodiment of a file access confirmation screen. The user allows the computer system 110 access by selecting the “allow” button 1008. FIG. 5E illustrates one exemplary embodiment of the completed profile photo upload. Optionally, the user may select the next button 1010 to enter a self-description for the profile (not shown).

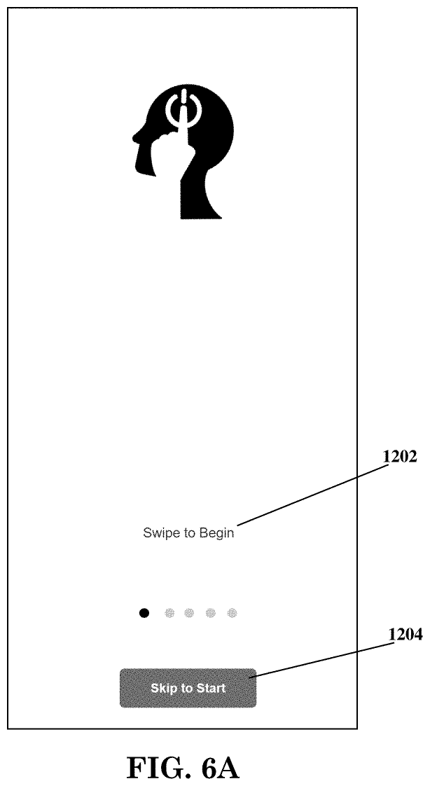

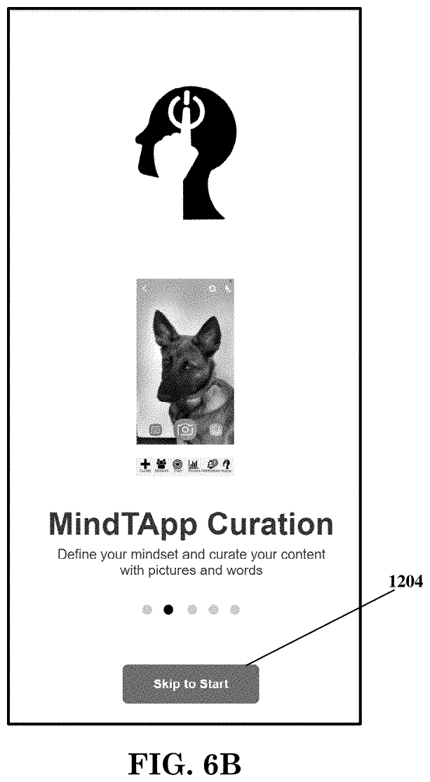

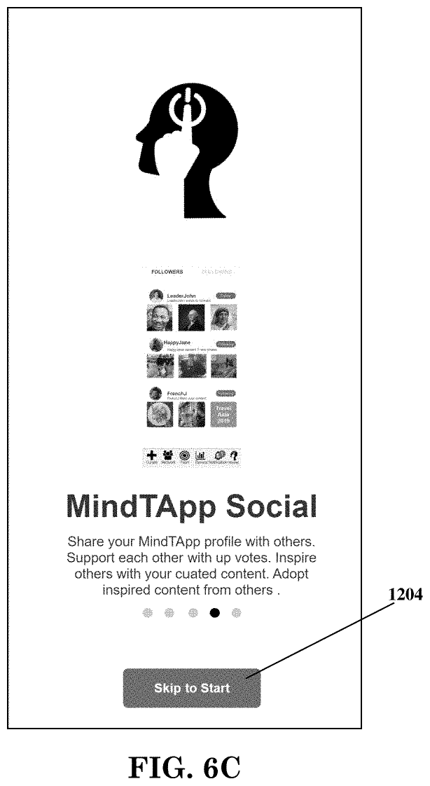

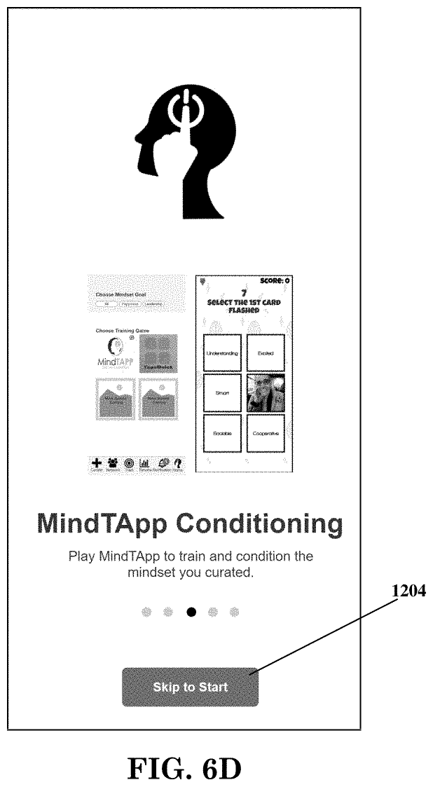

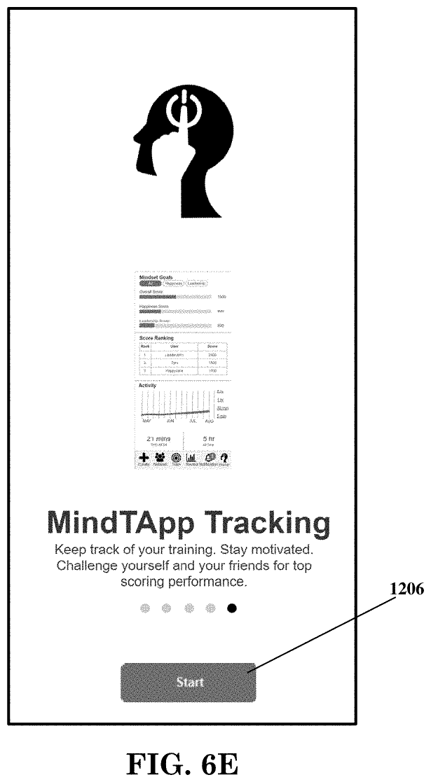

Upon completion of the user profile, the computer system 110 generates at step 402 of FIG. 3A a set of descriptions providing first-time users with an overview of the functionality of the application. The computer system 110 may void this step for returning users. FIGS. 6A to 6E illustrates screen views of one exemplary embodiment providing an overview of the functionality of the application to users. FIG. 6A illustrates a screen view of one exemplary embodiment of instructions prompting users to begin the overview. Users are instructed to “swipe to begin” 1202 the overview. FIG. 6B illustrates a screen view of one exemplary embodiment describing the function for curating mindset content. FIG. 6C illustrates a screen view of one exemplary embodiment describing the function for engaging social network. FIG. 6D illustrates a screen view of one exemplary embodiment describing the function for training mindset with conditioning games. FIG. 6E illustrates a screen view of one exemplary embodiment describing the function for reviewing performance.

Upon completing the overview of the functionality of the application, users may access the functionality of the application by selecting the “Start” button 1206 on the last screen of the overview FIG. 6E. Users may skip the overview by clicking the “Skip to Start” button 1204 on any of the screens of the overview (FIGS. 6A to 6D).

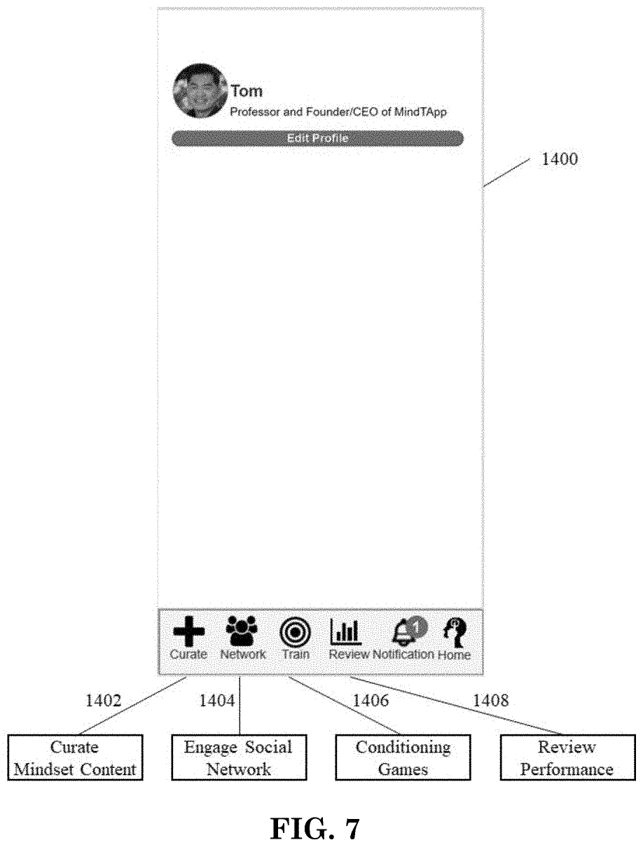

Upon selecting the “Skip to Start” 1204 or “Start” 1206 button, the computer system 110 performs the steps in flow diagram 400 shown in FIG. 3A and generates, at step 404, the profile screen 1400 shown in FIG. 7. The profile screen provides users with the following functional module buttons:
a. mindset curation 1402;
b. social network 1404;
c. conditioning games 1406; and
d. performance review1408.

User-Centric System and method for Curating User-Unique Content

Mindset training utilizing Associative Conditioning procedures involves the specification of a mindset training goal (e.g., happiness) and the identification of content that symbolically represent that mindset goal (e.g., photographs of sunshine or puppies that naturally induce positive emotions serve as symbolic representations for the domain of happiness). Prior art takes a professional-centric (pedagogical) method where users are dependent on skilled professionals (e.g., scientists, researchers, clinicians, application developers, etc.) to specify the mindset domain for intervention, and to generate, test, and validate content to determine its relevance for a general audience. Consequently, existing training interventions cover only a limited range of mindset domains. The exemplary embodiments of this disclosure reflect implementation improvements of prior art with a user-centric (andragogical) method where users are in charge of their mindset training, permitting users to develop their own interventions by specifying their mindset training goals and curating the corresponding mindset content. A user-centric method solves the problem of limitations in available interventions because users are in charge and can develop their own interventions for any mindset domain.

The exemplary embodiments of this disclosure solve the unidentified problem of the need for user-unique content for mindset training efficacy. Prior art teaches the necessity for skilled professionals to curate, test, and validate content, indicating that training interventions may not be possible without the guiding hand of a skilled professional. Moreover, Tsoneva emphasizes users should remain unaware of the guiding influence of skilled professionals because users may react to being manipulated towards a predetermined desired behavior. Typically, skilled professionals pilot test generic content to identify those that achieve acceptable consensus from a sample of users (e.g., 70% agreement vs. 30% disagreement), with the goal of identifying universal content that can represent a particular mindset for a general audience.

The exemplary embodiments of this disclosure bypasses the time consuming process of skilled professional-centric content curation because users curate content that are germane and specific to their mindset training goals. In particular, research results show that users' sense of connection to mindset content affects training efficacy. Training efficacy increases the more that mindset content accurately reflects users' mindset goals. Thus, mindset training is most efficacious when content is germane and specific to users. Generic content, for which users do not have a personal sense of connection, may be less effective in engaging users.

For example, providing a user training for a happiness mindset goal with a generic photo of a puppy may generally elicit a positive state (e.g., for someone belonging to the 70% sample in the above example). However, providing the same user with a photo of the user's own pet puppy (user-unique content) will elicit a higher happiness state. Moreover, a generic photo of a puppy may be irrelevant for some users (e.g., for those belonging to the 30% sample in the above example) because dogs do not represent nor bring happiness or dogs may represent a different mindset domain (e.g., a fearful mindset for users with negative experiences with dangerous attack dogs). Because content can represent different mindset domains to different users, the accuracy and validity of content representation resides with the unique user. Thus, whereas prior art teaches that skilled professionals should curate generic content for a general audience, the embodiments of this disclosure teach that mindset content is most efficacious when they are germane and specific to individual users, namely user-unique content.

Exemplary embodiments of this disclosure teach a system and method for representing individual users' mindset goals with visual, auditory, gustatory, olfactory, and tactile content representations, or a combination thereof. For example and without limitations, embodiments of this disclosure teach the use of content to represent individual users' unique mindset, involving motivations, attitudes, states, and traits. In doing so, the exemplary embodiments of this disclosure increase the precision of mindset representation and consequently, the efficacy of training.

Moreover, the exemplary embodiments of this disclosure teach a method with user-unique content that eliminates the requirement for two or more stimuli (e.g., CS—Conditioned Stimulus, and US—Unconditioned Stimulus) found in the typical Associative Conditioning paradigm of prior art. This multiple-stimuli method of prior art involves a pairing a first CS (e.g., content representing a mindset domain) with a second US (content representing a positive or negative evaluation). In contrast, the exemplary embodiments of this disclosure teach a single-stimulus method with user-unique content that simultaneously represents the CS and the US, e.g., a mindset domain along with its associated (positive or negative) characteristic.

For example, improving a user's happiness level in a typical Associative Conditioning procedure of prior art involves pairing two stimuli: 1) headshots of the user with 2) generic photos of positive stimuli (e.g., sunshine, puppy, the word “happy,” etc.—content that generally elicit a positive state in a general audience). Thus, the first stimulus photo (CS) represent the user (headshot of the user) and the second stimulus photo (US) represent happiness (e.g., generic image of a puppy). In contrast, a single-stimulus method involves user-unique content representing happiness that is personally relevant to the user (e.g., photos of the user's children, pet puppy, favorite hobby, etc.). In the single-stimulus method, the content (e.g., a photo of the user's actual pet puppy) simultaneously serve as a representation for the user (as the puppy's owner, the user is inherently represented within the puppy photo) and happiness (the user's actual pet puppy is a source of happiness). Representation of the user with the headshot photo is not needed in this case. Thus, exemplary embodiments of this disclosure teach a single-stimulus conditioning method with user-unique content that simultaneously represents a conditioned stimulus and an unconditioned stimulus (e.g., a mindset domain and its associated positive or negative characteristics), and thereby reduce complexity while improving efficiency that result in higher training efficacy.

The single-stimulus method for curating user-unique content underlies the exemplary embodiments of the mindset curation module 206 and social network module 208 of FIG. 2. Accordingly, users are in charge of their own mindset training by specifying their own mindset goals and curating the corresponding mindset content. This user-centric method reflects System 2 processes, where users' actions (e.g., specifying mindset training goals, and curating mindset content) operate in controlled (deliberate) and conscious fashion. These volitional actions of System 2 produce synergistic training effects when integrated with System 1 processes where users play the conditioning games that subconsciously train users in the mindset goal, as will be discussed later. Alternatively, the disclosed content curation (System 2) and conditioning game (System 1) process can be implemented independently for mindset training.

Finally, the curation of mindset content by the users serves as a form of conditioning to train users on that mindset. First, the conscious process of specifying mindset training goals and curating corresponding content (System 2) constitute a form of goal setting. The act of goal setting creates motivation for users to achieve their specified mindset training goals. Second, the process of curating mindset content on a consistent and repetitive interval (e.g., daily basis) creates attentional focus for users' specified mindset training goals. This attentional focus structures users' (daily) experience by increasing the likelihood and frequency that users are seeking and selectively attending to (daily) events and experiences that reflect their mindset goals. For example, users who have specified a happiness mindset goal are more likely to selectively attend to such corresponding events and experiences because the embodiments of this disclosure teach a system and method that prompts users to curate corresponding mindset content (e.g., capture a photo of happy moments and events with their mobile phone). Consistent with the tenets of Associative Conditioning, the curation of mindset content on a consistent and repetitive interval (e.g., daily basis) trains users in the mindset goal of interest by repetitively conditioning users with the contents of the mindset goal such that the repeated exposure result in that mindset goal becoming the dominant orientation.

### Exemplary Embodiment of Mindset Curation Module

Steps 406 to 414 of FIG. 3B show a simplified flow diagram illustrating exemplary processes performed by the mindset curation module 206 (shown in FIG. 2) in accordance with an exemplary embodiment of the disclosure. On execution of the Mindset Curation button 1402 (shown in FIG. 7), the computer system 110 generates for first-time users, at step 406, a set of instructions to facilitate users' identification of mindset goals (e.g., happiness) and means of representing the mindset goals (e.g., photos, text, sounds, smells, taste, vibrations, and videos). The computer system 110 may bypass step 406 for returning users who are familiar with the instructions.

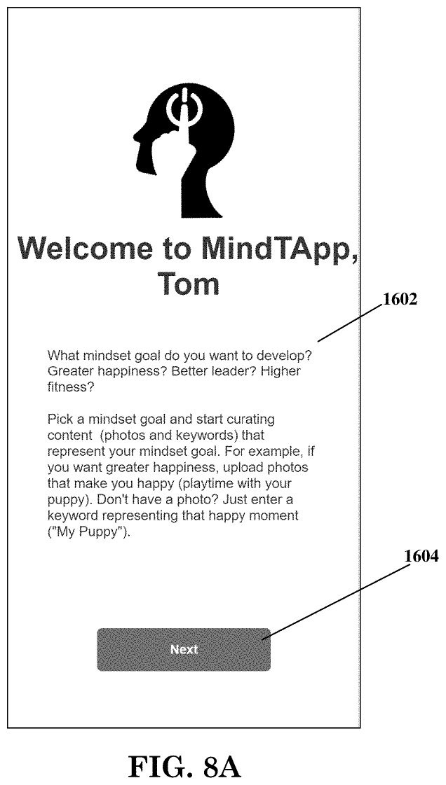

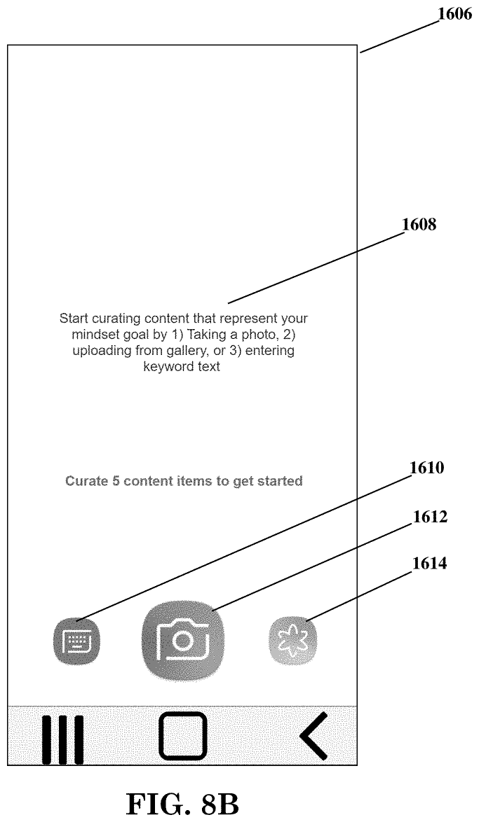

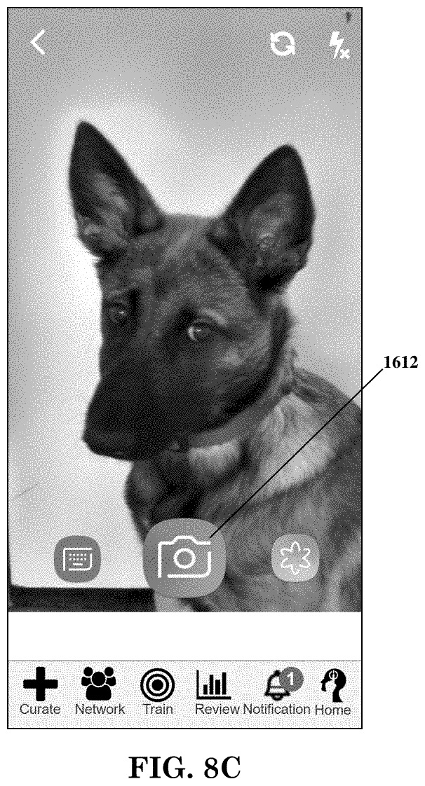

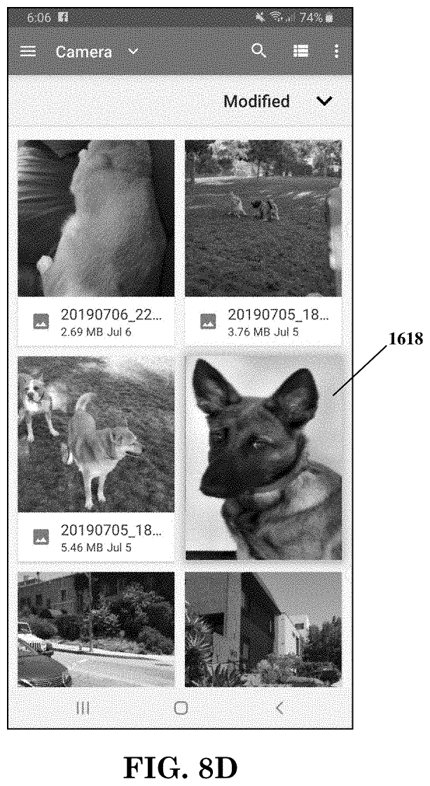

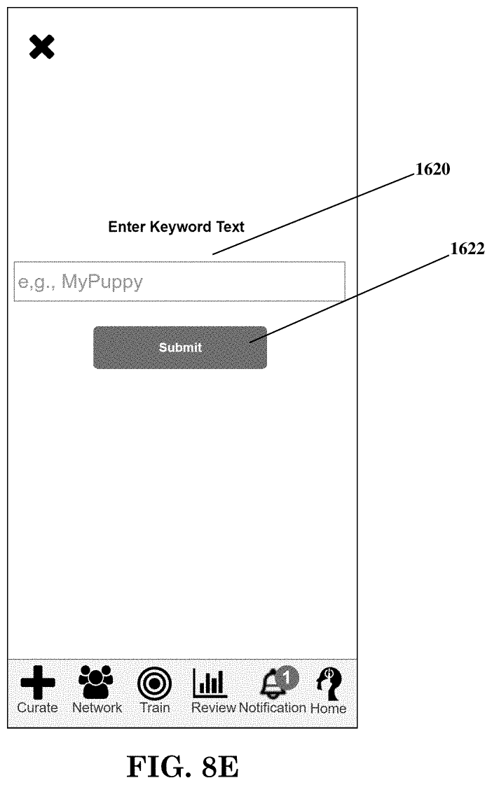

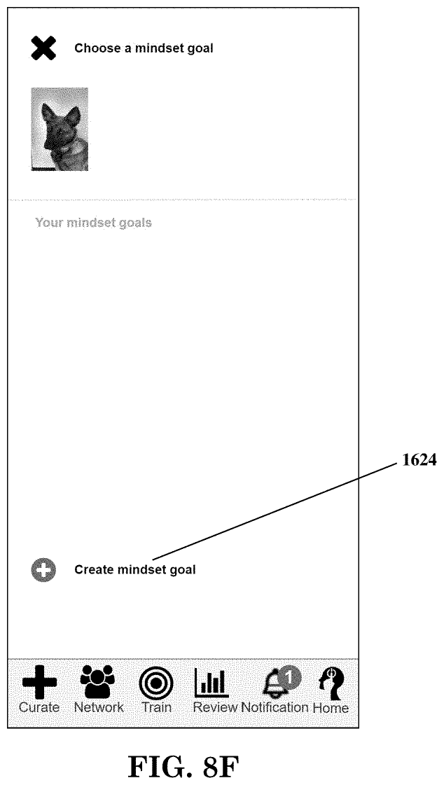

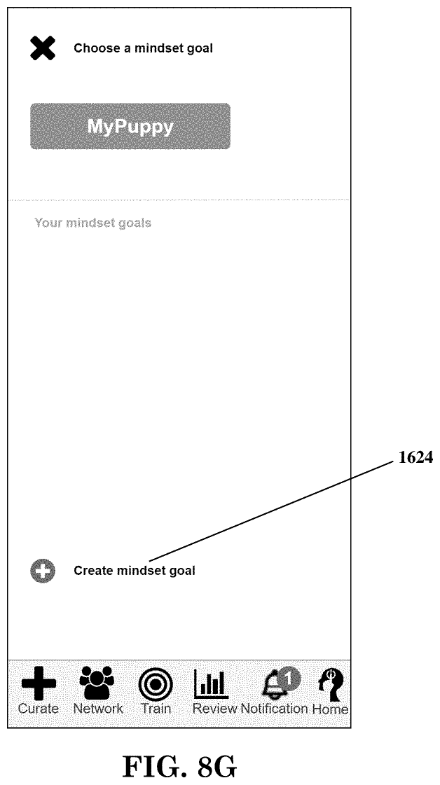

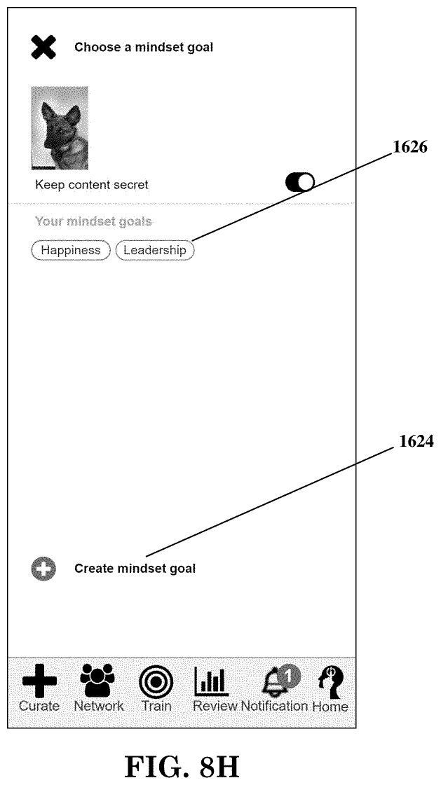

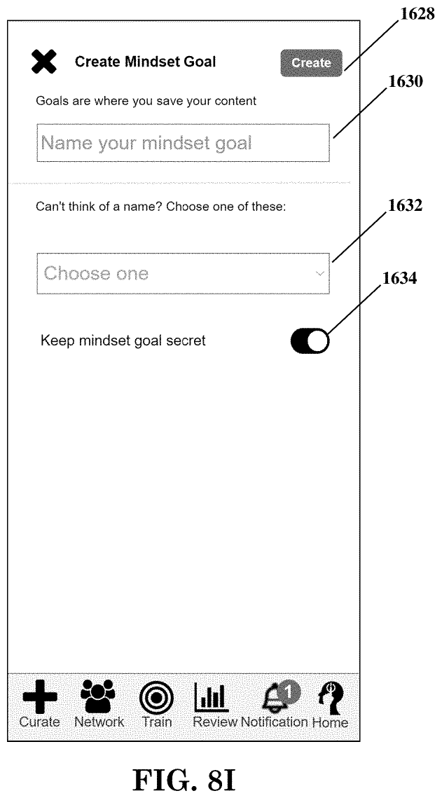

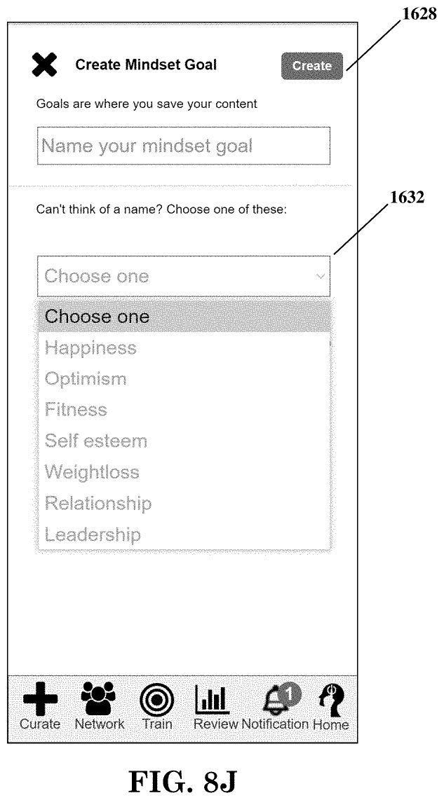

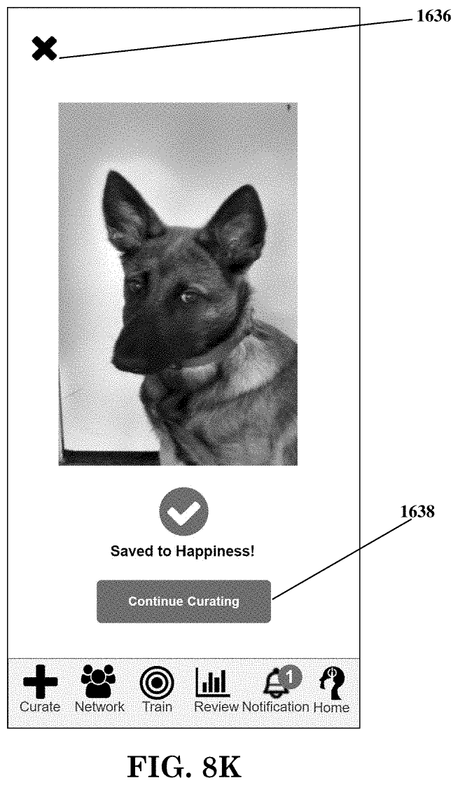

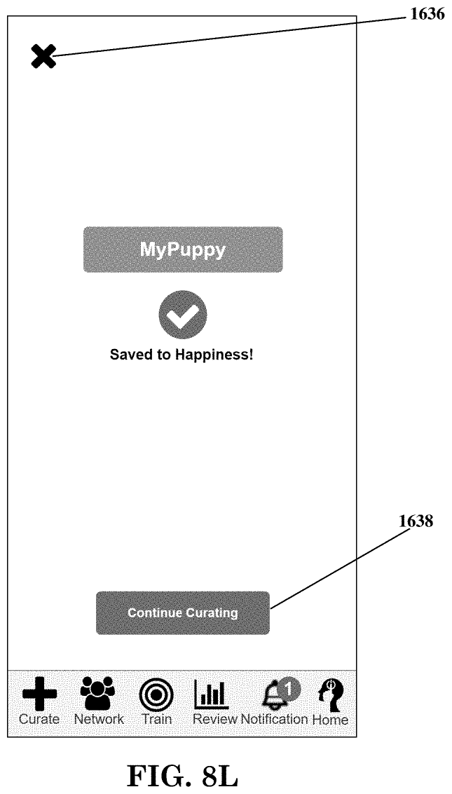

FIG. 8A illustrates a screen view of one exemplary embodiment of a set of instructions 1602 to guide users' identification of mindset goals and means of representing the mindset goals. Having read and understood the instructions, users selects the “Next” button 1604 to proceed.

Upon receiving user acknowledgment of the instructions, the computer system 110 generates, at step 408, a mindset curation graphical user interface 1606 for facilitating the curation of mindset content, as shown in FIG. 8B. For example and without limitations, the mindset curation GUI 1606 offers various means of capturing mindset content in the form of a photo or keyword, and associating each curated mindset content with a mindset goal by selecting from a list of mindset goal labels or inputting the name of the mindset goal. Some examples of mindset goals are “Happiness” (users who want to develop a happiness mindset) or “Leadership” (users who want to develop a leader mindset).

FIG. 8B illustrates a screen view of one exemplary embodiment of a set of instructions 1608 describing means for curating and uploading content that represent mindset goals. Users may upload content by selecting the keyboard function button 1610 to enter keywords (that describe the content), the camera function button 1612 to take a photo, or the photo library function button 1614 to select a photo. Optionally, users may upload content from their social network accounts (e.g., Facebook, Instagram, Twitter). For this illustration, the mindset content is curated via visual means (i.e., photos and keywords). However, mindset content can include a visual, an auditory, a gustatory, an olfactory, and a tactile representation, or a combination thereof.

FIG. 8C illustrates a screen view of one exemplary embodiment of a user selecting to upload mindset content using the camera function. In this example, the user is taking a photo of his or her puppy (the content item) by clicking on the camera function button 1612.

FIG. 8D illustrates a screen view of one exemplary embodiment illustrating a photo library that is accessed upon users selecting the photo library function 1614 to upload mindset content. In this example, the user is selecting the photo of his or her puppy (the content item) 1618 by clicking on the photo.

FIG. 8E illustrates a screen view of one exemplary embodiment illustrating a user selecting to upload mindset content using the keyboard function 1610 to enter a keyword for representing mindset content. The user is prompted with an entry field 1620 to enter a keyword. Selecting the entry field activates a keyboard for text entry (not shown). After entering the keyword e.g., “MyPuppy” (the content item), the user selects a “Submit” button 1622.

Having selected a mindset content item to upload, as illustrated in FIGS. 8C to 8E, the user is prompted to categorize the content item by associating it with a mindset goal, e.g., as shown in FIGS. 8F (for a photo upload) and 8G (for a keyword upload). For a returning user, the mindset goal listing 1626 provides options from which the user may choose, as shown in FIG. 8H. The options in the mindset goal listing 1626 are created by the user in previous rounds of curation. As shown, the exemplary embodiments of this disclosure reflect improved modifications of prior art whereby a single flexible and customizable intervention may accommodate one or multiple mindset goals for training, in comparison to prior art that can accommodate only a single mindset goal. For a first-time user or if the mindset content item does not have a corresponding mindset goal listing, the user is prompt to create a label for the new mindset goal by selecting the “Create mindset goal” button 1624 to continue to the mindset goal creation screen, as shown in FIG. 8I.

FIG. 8I illustrates one exemplary embodiment of a mindset goal creation screen. The user is prompted with two options to create a label for the mindset goal. The user may select the text entry field 1630 to enter a name (e.g., “Happiness”) for the mindset goal. Optionally, the user may decide to make the mindset goal open for viewing to members of a social network or keep it private by selecting the “Keep mindset goal secret” toggle button 1634. Alternatively, the user may select a name for the mindset goal from a drop down box 1632. Upon selecting the drop down box 1632, a list of common names for mindset goals are provided for the user's selection, e.g., as shown in FIG. 8J. In this example, the user may select “Happiness” from the drop down list. Having selected or entered a name for the mindset goal from the drop down box 1632 or text entry field 1630, respectively, the user selects a “create” button 1628 to continue.

Upon selecting the “create” button, the computer system 110 receives, at step 410, the user's input of the mindset content and its associated mindset goal. The computer system 110, in step 412, organizes the mindset content, e.g., with its respective mindset goal, and saves the mindset content into data storage 107. FIGS. 8K and 8L illustrate a screen view of one exemplary embodiment of step 412 for a photographic and textual (keyword) mindset content, respectively. As shown in FIG. 8K, the photo of a puppy (content item) has been uploaded and saved under the “Happiness” mindset goal. As shown in FIG. 8L, the keyword “MyPuppy” (content item) has been uploaded and saved under the “Happiness” mindset goal.

Optionally, the computer system 110 may require users to curate a minimum number of mindset content before proceeding further with the use of the application (e.g., before users are allowed to use the conditioning games functionality because the games may require the mindset content as stimuli in the conditioning games). The computer system 110, at step 414, determines if the minimum number of curated content required for game play has been met. If not, the computer system 110 prompts the user to continue curating content until the minimum number has been met (not shown). The user may continue to curate additional mindset content, e.g., to meet the minimum required, by selecting the “Continue Curating” button 1638, as shown in FIGS. 8K and 8L, which then returns the user to step 408. The user may also exit the process of curating content items by selecting the “X” button 1636, as shown in FIGS. 8K and 8L. Optionally, the mindset curation module 206 can be gamified whereby users can accumulate a predetermined amount of points for curating content (e.g., earn one point for each mindset content curated). For example and without limitations, a scoreboard can display to users the amount of points earned for content curation for each of their mindset goals, as well as a leaderboard displaying the ranking status of users within a social network for each mindset goal.

Mindset content is curated by users on a regular and ongoing basis (e.g., daily, weekly, etc.) via the mindset curation module 206. Thus, the content is dynamic and updated regularly, which has multiple benefits. First, it ensures that the content accurately, validly, and contemporaneously represents the mindset goal of users. Second, users' sense of connection to the mindset content is stronger with dynamic content that are novel, contemporaneous, and salient (vs. static and old content), which creates more user engagement.

### Exemplary Embodiment of Social Network Module

Steps 416 to 430 of FIG. 3C show a simplified flow diagram illustrating exemplary processes performed by the social network module 208 (shown in FIG. 2) in accordance with an exemplary embodiment of the disclosure. On executing the social network module button 1404 (shown in FIG. 7) for the first time, the computer system 110, at step 416, matches users with similar mindset goals within a social network. In one embodiment, the computer system 110 will generate matches based on the mindset goals resulting from steps 408 to 414. For example, the computer system 110 may generate matches based on users who selected or inputted “Happiness” to form a social network with members having a similar goal for developing a happiness mindset. Similarly, the computer system 110 may generate matches based on users who selected or inputted “Leadership” to form a social network with members having a similar goal for developing a leader mindset.

In another embodiment, the computer system 110 is configured to allow users to find matches using a keyword search function (not shown). For example, users may perform a search with the keyword “Happiness” to find like-minded others in pursuit of developing a happiness mindset. Similarly, users may perform a search with the keyword “leadership” to find like-minded others in pursuit of developing a leader mindset. Once matches have been found, the computer system 110 will display the matches for users. Users may choose to connect with each other by sending a connection request. If the connection request is accepted, users will be connected as a social network, which is displayed as part of step 418.

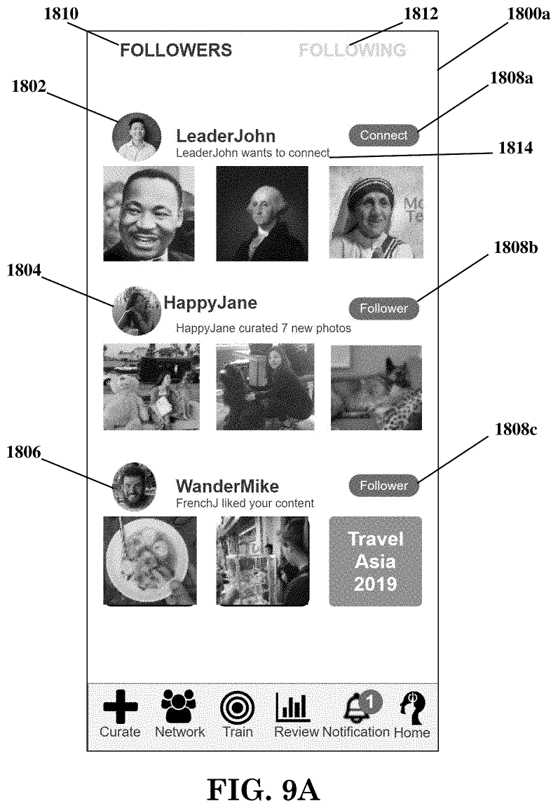

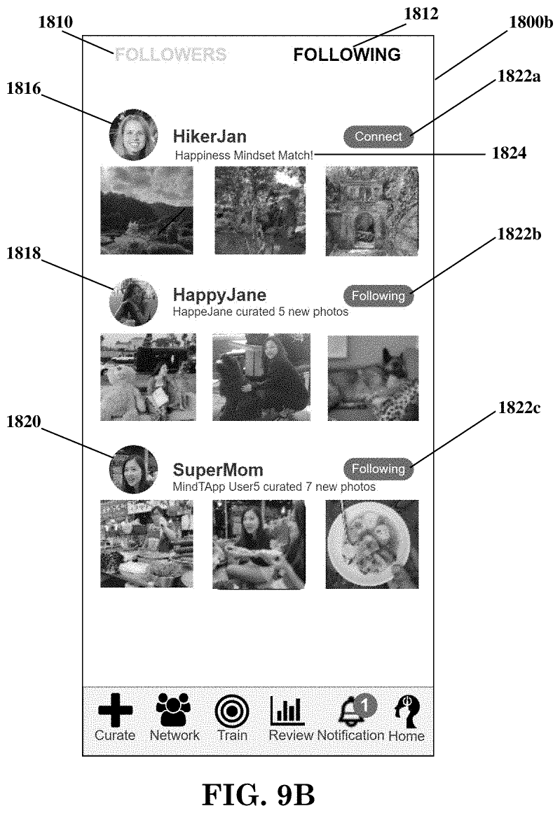

The computer system 110 generates, at step 418, a social network graphical user interface 1800a and 1800b for users to engage with other users in a social network. FIGS. 9A and 9B illustrates screen views of one exemplary embodiment of a social network GUI 1800a and 1800b displaying members connected to a user's social network and samples of curated mindset content for each of the members. FIG. 9A illustrates the user's followers within the social network. For this example, the user is being followed by HappyJane 1804, and WanderMike 1806. The followers status buttons 1808a to 1808c indicate the status for each follower. The user may change the status for each follower e.g., from “Follower” to “Block” (not shown) by clicking on the followers status buttons 1808b and 1808c to block a follower from continuing to follow the user. Optionally, the social network GUI 1800a may display others in the social network wanting to connect with the user, as shown in FIG. 9A with the example of LeaderJohn 1802 wherein the user receives a message alert 1814. In this example, the user may choose to connect with LeaderJohn 1802 by clicking on the followers status button 1808a, wherein it changes the status from wanting to “Connect” to “Follower.” The user may click on the followers status button 1808a one more time, wherein it changes the status to “Block” indicating the user choosing not to connect with LeaderJohn 1802. Users may switch between views of their followers and individuals they are following by clicking on the “Followers” 1810 and “Following” 1812 toggle at the top of the screen. After selecting the “following” toggle 1812, a user is led to a screen such as screen 1800b of FIG. 9B.

FIG. 9B illustrates others within the social network that the user is following. In the illustration 9B, the user is following HappyJane 1818 and SuperMom 1820. The “following status” button 1822a to 1822c indicates the status for each connected member. The user may change the status for each member, e.g., from “Following” to “Unfollow”(not shown) by clicking on the following status buttons 1822b and 1822c to stop following a follower in the social network. Optionally, the social network GUI 1800b may display others in the social network with matching mindset goals for which the user may want to follow, as shown in FIG. 9B with the example of HikerJan 1816, wherein the user receives a message alert 1824. In this example, the user may choose to connect with and follow HikerJan 1816 by clicking on the following status button 1822a, wherein it changes the status of a “Connect” suggestion to “Following.” The user may click on the followers status button 1822a one more time, wherein it changes the status to “Block” (not shown) indicating the user choosing not to connect with HikerJan 1816.

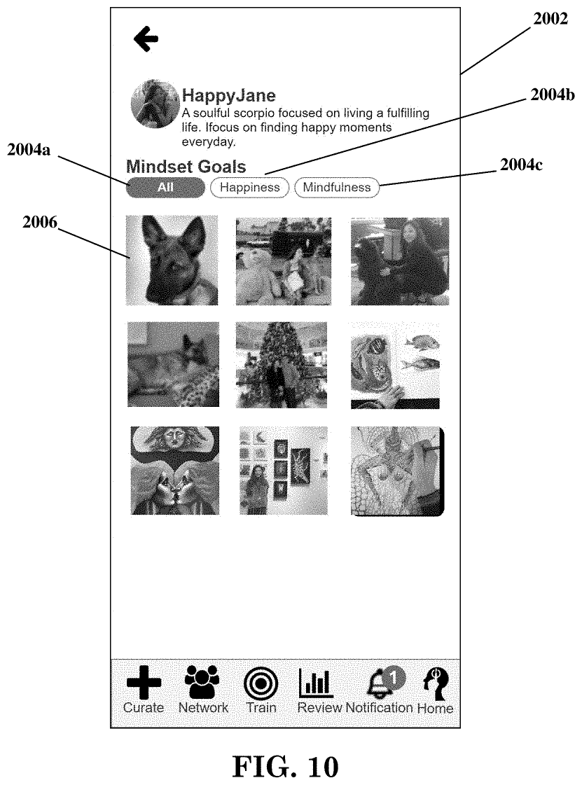

Users view the mindset content of the members of their social network by making a selection for that member. For example, in the illustration shown in FIG. 9B, a user clicks on the profile photo of HappyJane 1818 to go to the profile screen containing the mindset content of HappyJane. The computer system 110 receives, at step 420, user input for the selected member. Upon receipt of the input, the computer system 110, at step 422, generates and displays a member's profile screen 2002 containing that member's mindset content. FIG. 10 illustrates a screen view of one exemplary embodiment of a member's profile screen 2002 containing that member's mindset content. For the illustration shown in FIG. 10, the member profile screen 2002 containing the mindset content is configured to allow a user to choose to view all of the member's mindset content by selecting the “All” button 2004a, or to view the content for a specific mindset goal. For example, a user may view just the mindset content for the member's Happiness mindset goal by selecting the “Happiness” button 2004b. Similarly, a user may view just the mindset content for the member's Mindfulness mindset goal by selecting the “Mindfulness” button 2004c.

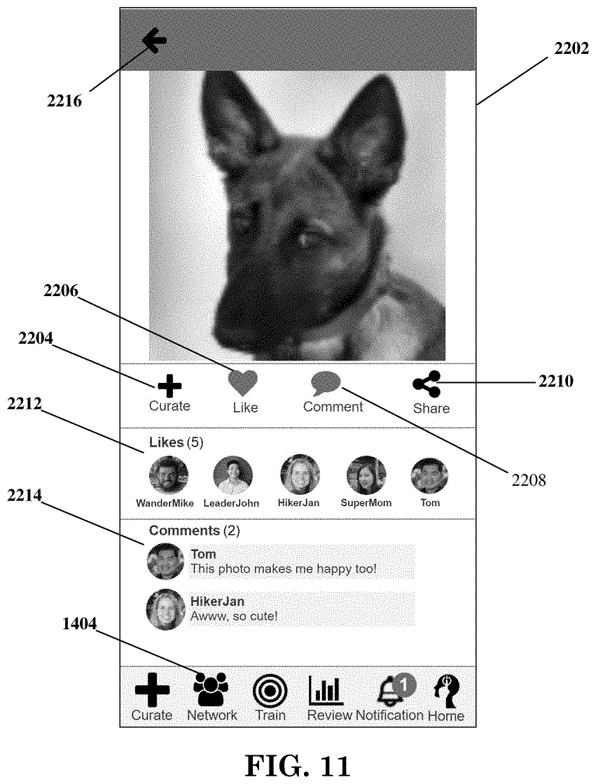

Users select a specific mindset content item to view its attributes. For example, in the illustration shown in FIG. 10, when a user clicks on the puppy photo 2006, the computer system 110 generates a profile screen for that specific mindset content item, as shown in FIG. 11. FIG. 11 illustrates a screen view of one exemplary embodiment of a profile screen for a specific mindset content item 2202 displaying its attributes.

Users may curate the specific mindset content at step 424, by selecting the Curate button 2204 in FIG. 11. After selecting the Curate button 2204, a user is prompted to categorize the specific mindset content by associating it with a user's existing mindset goal or creating a new mindset goal, e.g., similar to the description and illustrations for FIGS. 8F to 8L. The computer system 110 at step 426, receives user input for the specific mindset content and its associated mindset goal. The computer system 110, at step 428, organizes the mindset content e.g., by mindset goal, and saves the specific mindset content into data storage 107. Once saved, the user, at step 430, may continue to curate additional mindset content for a selected member by selecting the back-arrow button 2216 as shown in FIG. 11. Optionally, the user, at step 430, may continue to curate additional content items from other members in their social network, by selecting the social network module button 1404, which then returns the user to step 418.

Users curate mindset content from their social networks on a regular and ongoing basis (e.g., daily, weekly, etc.). Thus, the mindset content is dynamic and updated regularly, which has multiple benefits. First, it ensures that the content accurately, validly, and contemporaneously represents the mindset goal of users. Second, users' sense of connection to the mindset content is stronger with dynamic content that are novel, contemporaneous, and salient (vs. static and old content), which creates more user engagement.

Optionally, the content item profile screen 2202 provides a “like” button 2206 for users to indicate a favorable rating for the content item, a “Comment” button 2208 for users to post their own comments about the content item, and a “share” button 2210 for users to share the content item with others. Optionally, the content item profile screen 2202 provides a likes-display area 2212 for showing number of likes, along with the identity of users indicating a favorable rating for the content item. Optionally, the content item profile screen 2202 provides a comment-display area 2214 for showing existing comments for the content item. Optionally, the social network module 208 can be gamified whereby users can accumulate a predetermined amount of points for curating content from their social network, as well as rating, commenting on, and sharing content (e.g., earn one point for each rating, comment, share, or mindset content curated). For example and without limitations, a scoreboard can display to users the amount of points earned for curating content from their social network, rating, commenting on, and sharing content. Similarly, a leaderboard can display for users their ranking status for the aforementioned activities.

### Exemplary Embodiment of Conditioning Games Module

Steps 432 to 448 of FIG. 3D show a simplified flow diagram illustrating example processes performed by the conditioning games module 210 (shown in FIG. 2) in accordance with an exemplary embodiment of the disclosure. The exemplary embodiments illustrate a gamified Associative Conditioning procedure, which reflects System 1 processes. Consistent with System 1 processes, the conditioning games train the user in automatic and subconscious fashion via gameplay that do not require deliberate and conscious processing of the mindset content from users. Users merely play the games while indirectly being trained on the mindset content. Users may play the games alone or with others (e.g., other users in their social network). The conditioning games train users in the mindset goal of interest by repetitively conditioning users with the contents of the mindset goal such that the repeated exposure result in that mindset goal becoming the dominant orientation.

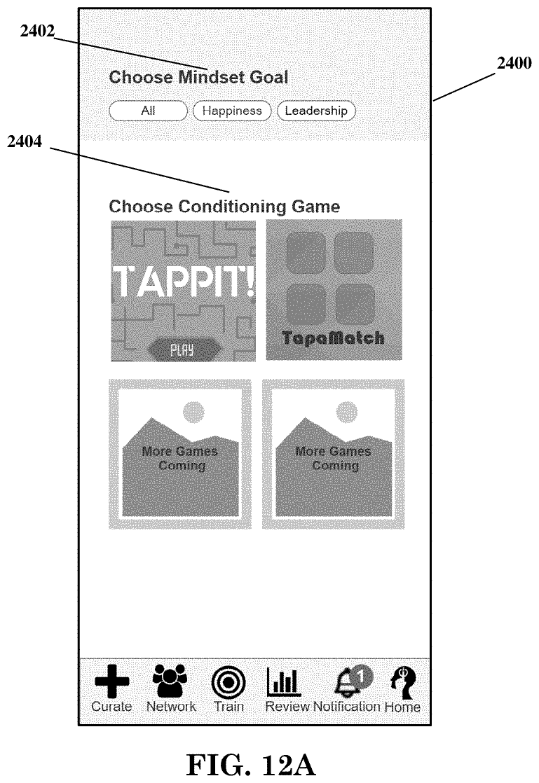

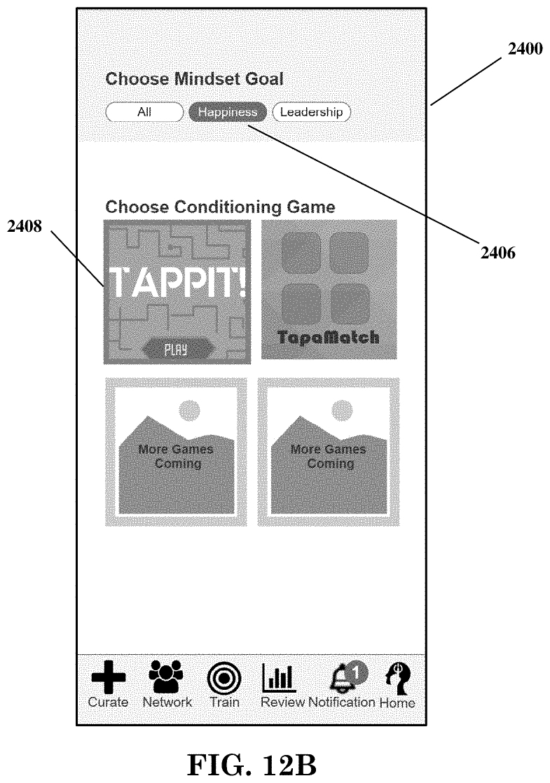

On execution of the conditioning games module button 1406 (shown in FIG. 7), the computer system 110 generates, at step 432, a conditioning games graphical user interface 2400, as shown in FIG. 12A. For the illustration 12A, the conditioning games GUI 2400 provides a list of available conditioning games 2404, as well as a list of mindset goals 2402 that users generated and saved during the content curation process in steps 408 to 414 and 418 to 430. Users may select any combination of conditioning games 2404 and mindset goals 2402. Additional conditioning games may be added on an ongoing basis via plugin or API and hosted on the computer system 110.

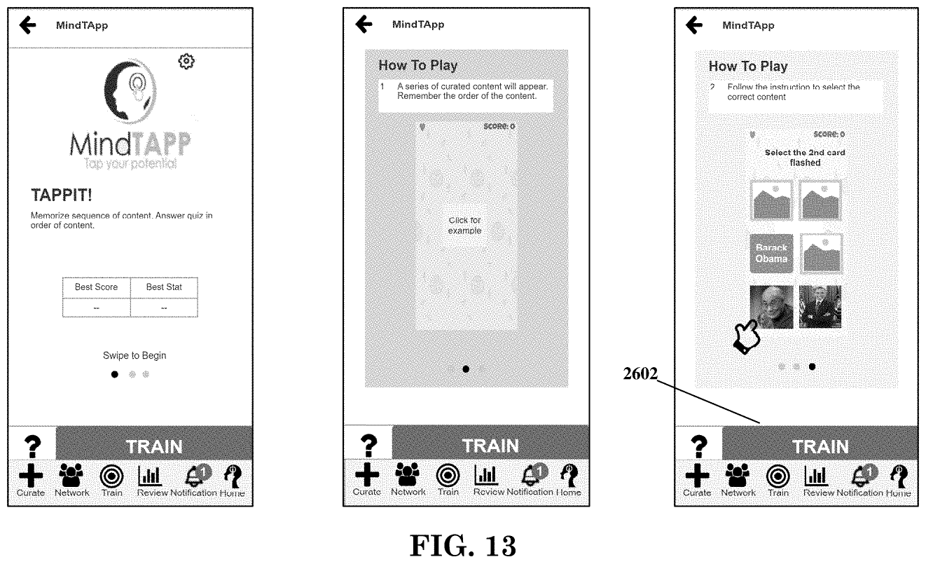

FIG. 12B illustrates a screen view of one exemplary embodiment wherein a user has selected for conditioning: 1) the mindset goal “happiness” 2406 with 2) the Tappit conditioning game 2408. The computer system 110 receives, at step 434, user input for the game attributes. On receiving the game attributes, the computer system 110 launches the game and provides instructions for the selected conditioning game in step 436. For example and without limitations, FIG. 13 illustrates a sequence of screen views of one exemplary embodiment of a set of instructions for a conditioning game. Different conditioning games 2404 will provide different instructions.

After receiving input from users via the selection of the Train button 2602, as shown in FIG. 13, the computer system 110 advances users to the game at step 438. The computer system 110 pulls the mindset content for the mindset goal selected by users in the conditioning games GUI 2400 (e.g., happiness 2406 of FIG. 12B) and transforms the mindset content into game stimuli for the selected conditioning game (e.g., Tappit Conditioning game 2408 of FIG. 12B). The game stimuli are comprised of the mindset content items generated via the mindset curation module 206 in steps 408 to 414 and via the social network module 208 in steps 418 to 430.

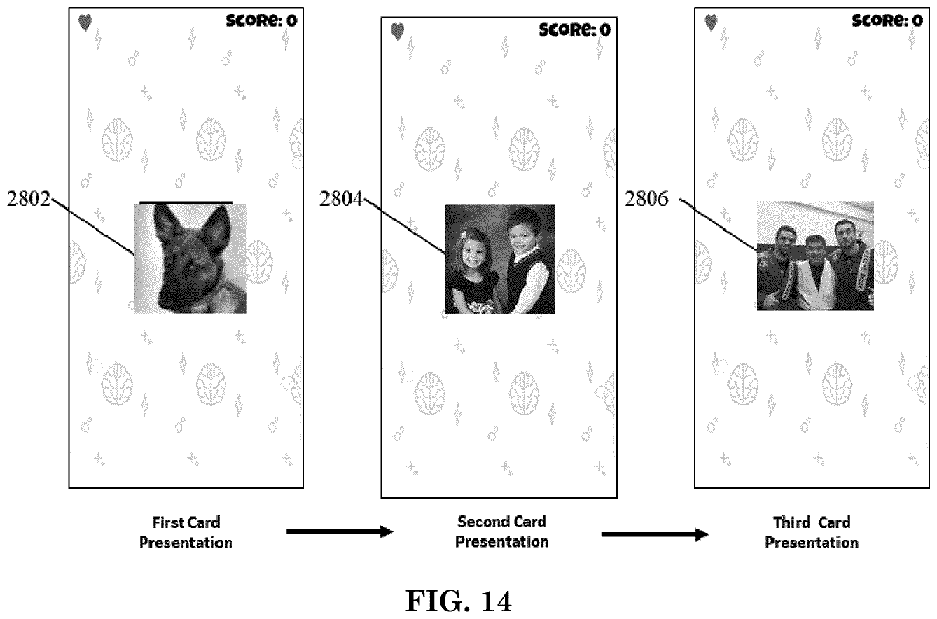

The computer system 110 selects a random set (e.g., 3 items) of the game stimuli and presents them sequentially for a predetermined amount of time (e.g., presents each game stimuli for 1 second). FIG. 14 illustrates an exemplary embodiment of a conditioning game designed to increase happiness in users by presenting user-unique game stimuli representing happiness. As noted earlier, the single-stimulus method permit users to curate user-unique mindset content (game stimuli) that simultaneously represents a conditioned stimulus and an unconditioned stimulus (e.g., a single stimulus photo that simultaneous serve as a representation for the user and happiness). In the example, a user is presented stimulus photos (game stimuli) of the user's happy moments, including a photo of the user's pet puppy 2802, children 2804, and favorite martial arts hobby 2806. Repetitive presentation of the user-unique stimuli simultaneous pairs and conditions users with their happiness moments causing them to view themselves as happy.

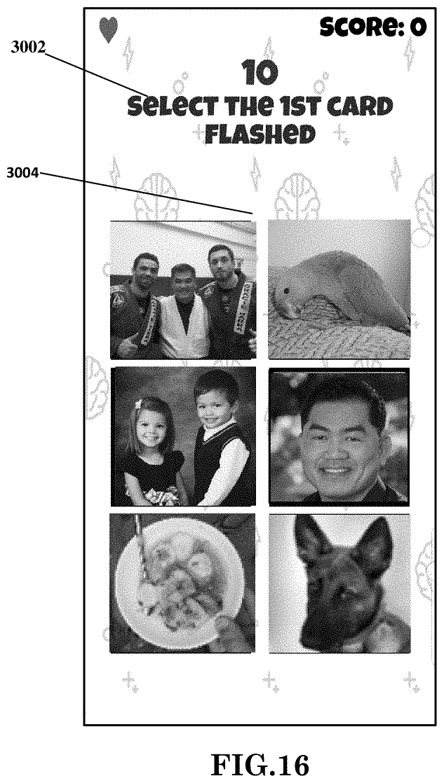

FIG. 14 illustrates a sequence of screen views of one exemplary embodiment of a conditioning game, showing a trial with a sequence of three stimulus photos wherein the first stimulus photo (user's pet puppy) 2802 is shown for 1 second, followed by a display of a blank screen for half a second (not shown); the second stimulus photo (user's children) 2804 is shown for 1 second, followed by a display of a blank screen for half a second (not shown); and the third stimulus photo (user's favorite martial arts hobby) 2806 is shown for 1 second, followed by a display of a blank screen for half a second (not shown). Following the presentation of stimulus photos, the computer system 110, at step 440, prompts the user to respond to a query, as shown in FIG. 16. FIG. 16 illustrates a screen view of one exemplary embodiment of a query screen displaying the query 3002. The computer system 110 prompts the user to respond by selecting an answer from a set of options in the answer choices portion 3004.

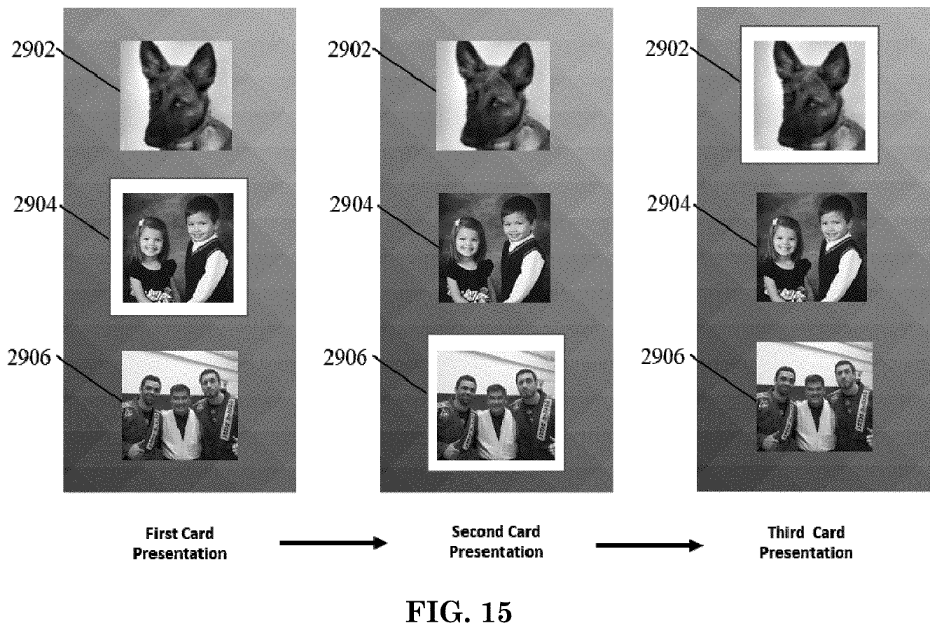

FIG. 15 further illustrates a sequence of screen views of another embodiment of a conditioning game utilizing the same game stimuli (stimulus photos) to increase happiness in users. In the example, the computer system 110 presents a user with stimulus photos (game stimuli) of the user's happy moments. Each stimulus photo is then highlighted with a white color border in an ordered sequence. In the example, the stimulus photo of the user's children 2904 is highlighted first; the stimulus photo of the user's favorite martial arts hobby 2906 is highlighted second; and the stimulus photo of the user's pet puppy 2902 is highlighted third. The computer system 110 then prompts the user to select (click on) the stimulus photos in the same order sequence that they were highlighted.

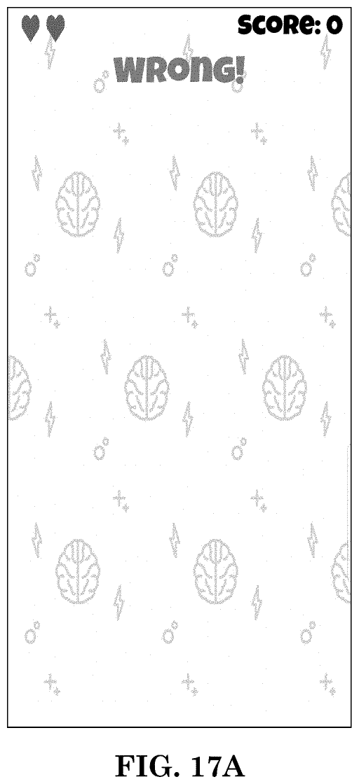

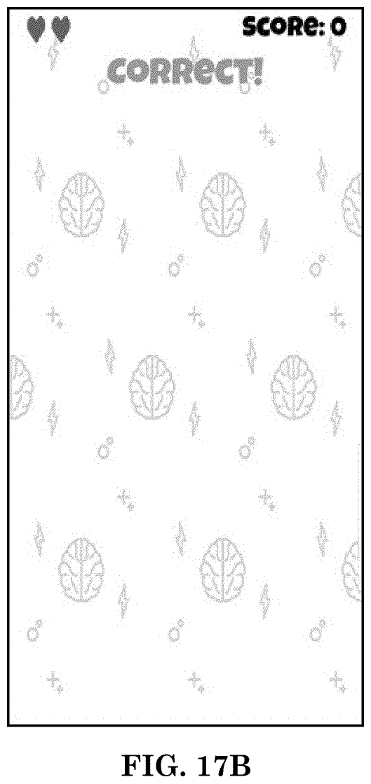

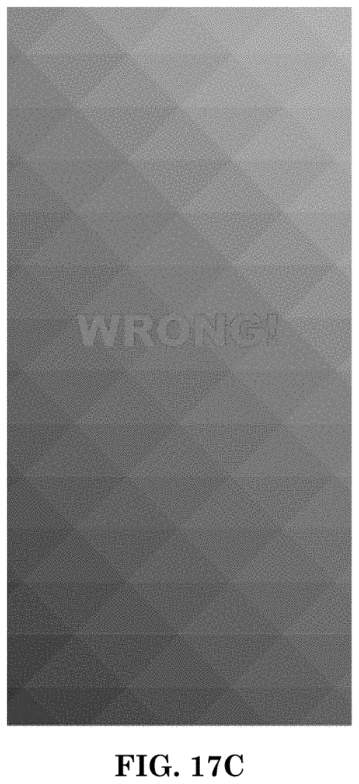

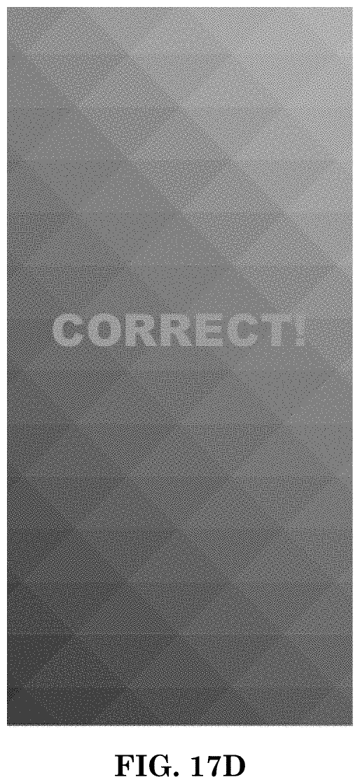

The computer system 110 receives the user's input at step 442 and records its speed and accuracy, wherein these data are used for calculating a user's performance score based on a predetermined algorithm (e.g., fast and accurate responses receive higher scores). The computer system 110 provides feedback to the user on the accuracy of the user's response at step 444, as shown in FIGS. 17A and 17B for the conditioning game illustrated in FIG. 14, and in FIGS. 17C and 17D for the conditioning game illustrated in FIG. 15. FIGS. 17A and 17C respectively illustrate screen views of exemplary embodiments of feedback for the user wherein the user provides an incorrect response to the query 3002 for the conditioning game illustrated in FIG. 14 and incorrect order sequence response for the conditioning game in FIG. 15. FIGS. 17B and 17D respectively illustrate screen views of exemplary embodiments of feedback for the user wherein the user provided a correct response to the query 3002 for conditioning game illustrated in FIG. 14 and correct order sequence response for the conditioning game in FIG. 15.

Users play at least one or more trials of the conditioning game sequence (steps 438 to 444). Each trial provides auditory and visual feedback, and reward points to indicate if the trial was performed correctly or incorrectly. The computer system 110, at step 446, progressively adjusts the difficulty level after each trial based on a predetermined algorithm.

After adjusting the difficulty level, the computer system 110 begins a new trial by returning users to step 438. For example, a user providing a correct response on a first trial sequence with three game stimuli, as shown in FIGS. 14 and 15, will advance to a more challenging second trial sequence with four game stimuli, followed by an even more challenging third trial sequence with five game stimuli, and so on.

In another embodiment, the game is configured to advance to more difficult trials based on users achieving a predetermined degree of performance on multiple trials. For example, users must achieve approximately 70% correct responses on ten trials consisting of three game stimuli before advancing to the next level of difficulty with trials consisting of four game stimuli, and so forth. Maintaining a relatively high level of success helps prevent frustration and encourages users to continue training. Optionally, the computer system 110 may update the level of difficulty with different parameters, for example and without limitations, such as the presentation speed of the game stimuli. Accordingly, the game stimuli may be presented at a faster rate of half a second instead of one second.

Users continue playing for as long as users wish, or until the game play reaches a major completion stage. Optionally, summary game metrics (points, levels, usage, progress) are shown to users during game play or at the end of each session (not shown). Optionally, users may also pause play or select another game at any time (not shown). Upon receiving user feedback, at step 448, the computer system 110 saves the game position so that users may return to the same position in a future session, and returns users to step 432 if users provide input to play another conditioning game, or the computer system 110 ends the conditioning game. Optionally, the conditioning games module 210 can be gamified whereby users can accumulate a predetermined amount of points for playing the various conditioning games (e.g., earn 10 point for each game session played), as well as for performance on the conditioning games. For example and without limitations, a scoreboard can display to users the amount of points earned for the number of games played, performance score for each of the different conditioning games, as well as a leaderboard displaying the ranking status of users within a social network for a specific conditioning game or across all conditioning games.

In sum, the exemplary embodiments of this disclosure reflect improvements over prior art whereby a single flexible and customizable intervention may accommodate one or multiple mindset goals for training, as well as provide multiple conditioning games, in comparison to the rigid systems and methods of prior art that can condition only a single mindset goal with a single conditioning game or task. Thus, users may train the same set of mindset goals in a variety of conditioning games, which enhances user engagement and training effectiveness. Moreover, a variety of conditioning games are easier to develop because game stimuli are derived and transformed by the computer system 110 from the same mindset content users curate.

Furthermore, the exemplary embodiments of this disclosure reflects improvements over prior art by producing synergistic effects in training by integrating System 1 and System 2 processes in a single training intervention. Prior art teaches that System 1 and System 2 are not compatible for integration, characterizing the dual thinking system as operating in opposing fashion. System 1 is characterized as operating in fast, automatic (involuntary) and subconsciously fashion. In contrast, System 2 is characterized as operating in slow, controlled (deliberate) and conscious fashion. For example, Greenwald claims that the mindset content of system 1 are implicit (subconscious) in nature and therefore not consciously accessible nor controllable by users. Similarly, Tsoneva claims that the effectiveness of Associative Conditioning procedures (System 1) would be reduced if users became consciously aware of its purpose or goal (System 2). Thus, System 1 processes have been characterized by prior art as being devoid of conscious goals, purpose, awareness and controllability for users. As such, prior art indicates that individuals lack the awareness, control, and motivational goal to be in charge of their mindset training. Consequently, prior art teaches a professional-centric method where users has little input or control over their mindset training.

Such claims are not supported by recent findings of scientific studies. Reviews of the scientific literature indicate that conscious awareness of the purpose and goals for an Associative Conditioning procedure is the most important factor for intervention efficacy. When individuals are consciously aware of the purpose and goals for an Associative Conditioning procedure, its effectiveness improves by 300%. These recent scientific findings have been extrapolated to devise a user-centric method disclosed in the exemplary embodiments of this disclosure.

The exemplary embodiments of this disclosure reflect a user-centric method where users are in charge of developing their mindset training by specifying their mindset training goals and curating the corresponding mindset content. Accordingly, users are consciously aware of the goal and purpose of their mindset training and the Associative Conditioning procedure involved in the conditioning games. The conscious process of specifying mindset training goals and curating corresponding content (System 2) constitute a form of goalsetting that enhances the effectiveness of the subconscious procedure of Associative Conditioning (System 1). Thus, the exemplary embodiments of this disclosure reflects improvements over prior art by producing synergistic training effects by integrating System 1 and System 2 processes in a single training intervention.

### Exemplary Embodiment of Review Performance Module

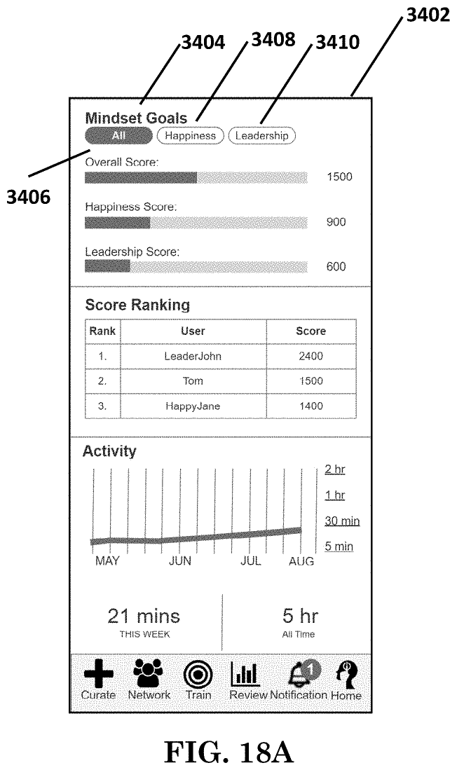

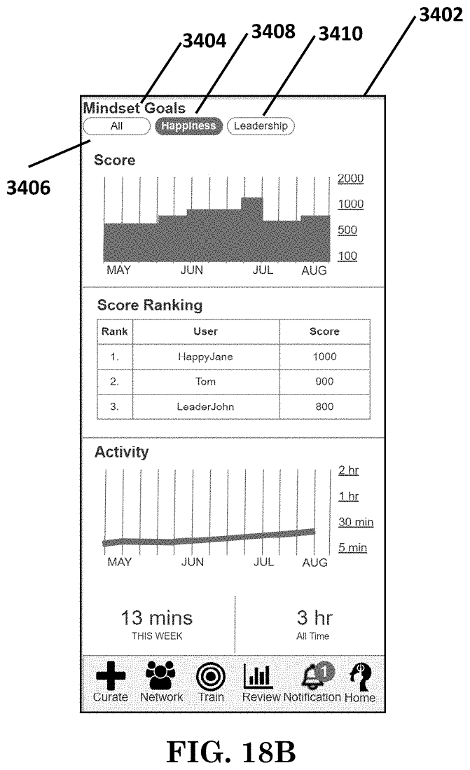

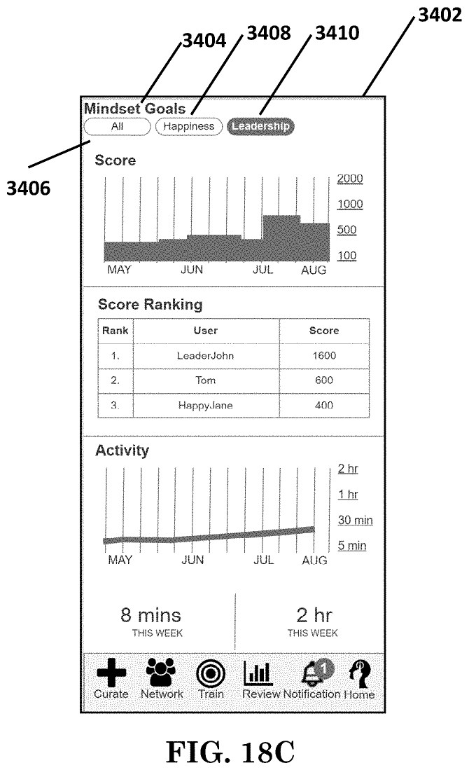

Steps 450 to 456 of FIG. 3E show a simplified flow diagram illustrating example processes performed by the performance review module 212 (shown in FIG. 2) in accordance with an exemplary embodiment of the disclosure. On execution of the performance review module button 1408 (shown in FIG. 7), the computer system 110 generates, at step 450, a performance review graphical user interface 3402, as shown in FIGS. 18A to 18C, comprising of performance metrics that has been stored in the database 107. The performance review GUI 3402 is configured to allow users to select for review the performance metrics for all of their mindset goals or to review the performance metrics for a single mindset goal by selecting among the options in the mindset goal selection section 3404. In this example, users may choose to view the performance metrics for all of their mindset goals by selecting the “All” button 3406 or view the performance metrics for each specific mindset goal by selecting the “Happiness” button 3408 or the “Leadership” button 3410.

The Computer System 110 receives, at step 452, user input for the performance review metrics. On receiving user input, the computer system 110 generates and displays the selected performance metrics at step 454. Users, at step 456, may continue to review the performance metrics for another mindset goal by making another selection from among the options in the mindset goal selection section 3404, wherein the computer system 110 receives users' input at step 452, and generates and displays the selected performance metrics at step 454. When users select the “All” button 3406, the computer system 110 generates the performance review GUI 3402 displaying the performance metrics for all of users' mindset goals, as shown in FIG. 18A. When users select the “Happiness” button 3408, the computer system 110 generates the performance review GUI 3402 displaying the performance metrics for users' happiness mindset goal, as shown in FIG. 18B. When users select the “Leadership” button 3410, the computer system 110 generates the performance review GUI 3402 displaying the performance metrics for users' leadership mindset goal, as shown in FIG. 18C.

The performance metrics may comprise various types, for example and without limitations, including:
a. performance score data for a specific game;
b. a summary performance score for all games;
c. number of games played;
d. history of scores for games played;
e. leaderboard with performance rankings comparing multiple users;
f. time spent playing a specific game across a plurality of time periods (e.g., day, week, month, year);
g. time spent playing across all games across a plurality of time periods (e.g., day, week, month, year);
h. number of curated mindset content for all mindset goals or a specific mindset goal;
i. amount of time spent curating mindset content for all mindset goals or a specific mindset goal;
j. number of likes and comments received and/or shared; and
k. number of content items shared or curated by others.

Optionally, the performance review module 212 (shown in FIG. 2) can be gamified whereby users can accumulate a predetermined amount of points on the basis of performance metrics. For example and without limitations, a scoreboard can display to users the amount of points earned for each performance metric, as well as a leaderboard displaying the ranking status of users within a social network for each performance metric.

Advantages
From the description above, a number of advantages of some embodiments of the disclosed mindset training system and method become evident as follows:
A. User-Centric Method. The exemplary embodiments of this disclosure reflect implementation improvements of prior art with a user-centric (andragogical) method where users are in charge of developing their own training by specifying their own mindset training goals and curating the corresponding mindset content. A user-centric method solves the problem of the dearth of available interventions because users are in charge and can develop their own interventions for any mindset domain.
B. User-Unique Content. The exemplary embodiments of this disclosure solve the unidentified problem of the need for user-unique content for mindset training efficacy. The exemplary embodiments of this disclosure bypass the time consuming process of skilled professional-centric content curation, which also has less training efficacy. Mindset training works best when the content is germane and specific to users.
C. Precise Representation of User Mindset. The exemplary embodiments of this disclosure teach a system and method for representing individual users' unique mindset, involving motivations, attitudes, states, and traits. Users' unique mindset may be represented with visual, auditory, gustatory, olfactory, and tactile content, or a combination thereof. In doing so, the exemplary embodiments of this disclosure increase the precision of mindset representation and consequently, the efficacy of training.
D. Dynamic Mindset Content. Mindset content is curated by users on a regular and ongoing basis via the mindset curation module 206 and social network module 208. Thus, the content is dynamic and updated regularly, which has multiple benefits. First, it ensures that the content accurately, validly, and contemporaneously represents the mindset goals of users. Second, users' sense of connection to the mindset content is stronger with dynamic content that are novel, contemporaneous, and salient (vs. static and old content), which creates more user engagement.
E. Flexible and Customizable System and Method. The exemplary embodiments of this disclosure reflect improved modifications of prior art whereby a single flexible and customizable intervention may accommodate one or multiple mindset goals for training, as well as provide multiple conditioning games, in comparison to the systems and methods of prior art that can train only a single mindset goal with a single conditioning game or task. Thus, users may train the same set of mindset goals in a variety of conditioning games, which enhances user engagement and training effectiveness. Moreover, conditioning games are easier to develop and integrate (e.g., via plugin or API) because game stimuli are derived and transformed by the computer system from the same mindset content curated by users.
F. Synergistic Application of Dual Thinking System. Mindsets are formed and changed based on a dual thinking system, namely System 1 and System 2. Accordingly, mindset training is most efficacious when both systems are engaged. The exemplary embodiments of this disclosure reflect improvements over prior art by producing synergistic training effects by integrating System 1 and System 2 processes in a single training intervention. The conscious process of specifying mindset training goals and curating corresponding content (System 2) constitute a form of goalsetting and attentional focus that enhances the effectiveness of the subconscious procedure of Associative Conditioning (System 1).
G. Synergistic Engagement Features. The exemplary embodiments of this disclosure engage user motivation with a gamification system of rewards, involving a plurality of game activities (e.g., content curation, conditioning games, etc.), performance review metrics (e.g., time spent playing games, game scores, number of mindset content curated), and social network engagement (e.g., number of likes and comments received or shared, number of content items shared or curated by others, etc.). Moreover, the social network motivates users through the support received from others (e.g., receiving supportive comments, benefiting from the motivational mindset contents from other members of social network, etc.).
H. Single Stimulus Conditioning. The exemplary embodiments of this disclosure teach a user-centric method that eliminates the requirement for two stimuli found in the typical Associative Conditioning paradigm of prior art. Specifically, the exemplary embodiments of this disclosure teach a single-stimulus method with user-unique content that simultaneously represents a conditioned stimulus and a unconditioned stimulus (e.g., a mindset domain and its associated positive or negative characteristics), and thereby reduce complexity while improving efficiency that result in higher training efficacy.

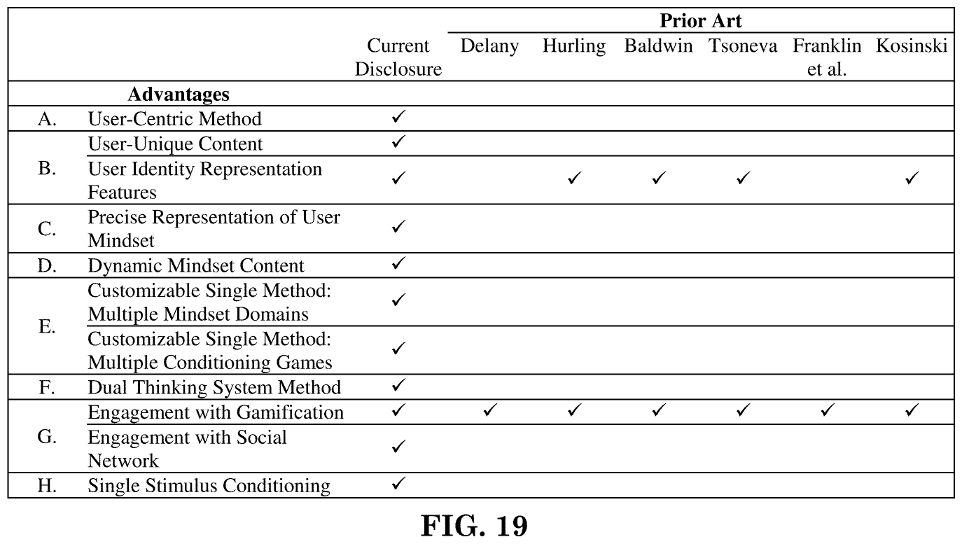

These advantages of the disclosed embodiments are distinct from prior arts as summarized in the comparison table shown in FIG. 19. Franklin et al. in FIG. 19 refers to an article entitled “A brief mobile app reduces nonsuicidal and suicidal self-injury: Evidence from three randomized controlled trials” by J. C. Franklin, K. R. Fox, C. R. Franklin, E. M. Kleiman, J. D. Ribeiro, A. C. Jaroszewski, J. M. Hooley, and M. K. Nock, published in 2016 in the Journal of Consulting and Clinical Psychology, 84(6), 544-557. Kosinski in FIG. 19 refers to an article entitled “A brief mobile evaluative conditioning app to reduce body dissatisfaction? A pilot study in university women” by T. Kosinski, published in 2019 in the journal Frontiers in Psychology, 10, 2594.

Conclusion, Ramifications, And Scope

As seen, at least one embodiment of the mindset training system and method provides effective means for mindset training with gamification that 1) permits users to be in charge of their own mindset training interventions, 2) permits users to represent their unique mindset, involving motivations, attitudes, states, and traits, via multiple modalities including, visual, auditory, gustatory, olfactory, and tactile, or a combination thereof, 3) permits users to specify and input one or more mindset training goals, 4) permits users to curate mindset content that is germane and specific to users, 5) permits users to curate user-unique content that simultaneously represents a conditioned stimulus and a unconditioned stimulus (e.g., a mindset domain and its associated positive or negative characteristics), 6) permits users to train their mindset goals via the curation of mindset content 7) transforms user-unique content into game stimuli by the computer for use as inputs in conditioning games, 8) permits users to train their mindset goals by playing a variety of conditioning games, 9) provides social network functionalities for user engagement, motivation, and support, and 10) permits users to track performance and review progress for their mindset training.

Furthermore, the mindset training system and method have the following additional advantages:
it solves the problem of the dependency on skilled professionals to develop mindset training interventions;
it solves the problem of the dearth of available mindset training interventions;
it enhances the efficacy of mindset training with the use of user-unique content;
it enhances the efficacy of mindset training via the accurate, valid, and contemporaneous representation of users' mindset with visual, auditory, gustatory, olfactory, and tactile content, or a combination thereof;
it permits the training of a host of mindset domains, including and without limitations, motivations, attitudes, states, and traits;
it provides users with means of curating mindset content on a regular and ongoing basis so that they accurately, validly, and contemporaneously represents the mindset goal of interest, which creates a stronger sense of connection to the mindset content and thus more user engagement;
it permits users to train multiple mindset goals simultaneously in a single training intervention;
it provides a variety of conditioning games by configuring dynamic combinations of conditioning games with mindset goals;
it provides a means of integrating additional conditioning games on an ongoing basis to sustain user engagement;
it enhances mindset training efficacy by integrating System 1 and System 2 processes in a single training intervention;
it enhances user engagement with a gamification system of rewards involving a plurality of conditioning games, performance review metrics, and social network engagement; and
it improves training efficiency and efficacy with a single stimulus conditioning method involving user-unique content that simultaneously represents a conditioned stimulus and an unconditioned stimulus (e.g., a mindset domain and its associated positive or negative characteristics).

While the description contains many specificities, these should not be construed as limitations on the scope, but rather as an exemplification of several embodiments thereof. Many other variations are possible, including:
Marketplace for Conditioning Games. The exemplary embodiments of this disclosure provide a means of integrating additional conditioning games on an ongoing basis. Accordingly, a marketplace can be established that permit software developers to develop conditioning games that can be integrated via plugin or API;
Marketplace for Curated Mindset Content. The exemplary embodiments of this disclosure teach a user-centric system and method for curating user-unique content. Optionally, the exemplary embodiments of this disclosure permit a professional-centric method whereby skilled individuals may curate content for various mindset domains for purchase to users who do not want to curate their own user-unique content;
User Psychological Profile. The user profile and mindset goals created by users represent their psychological profile, enabling others to understand the underlining psychological makeup of users. For example, a user profile with “happiness” and “leadership” as mindset goals (i.e., a user's psychological profile) reflects someone with an orientation towards cultivating happiness and being a leader. Similarly, a user profile with “jogging” and “organic diet” as mindset goals reflects someone with an orientation towards health and fitness. User psychological profiles may be utilized to provide insights in a variety of domains such as product purchases, branding preferences, voting behaviors, dating preferences, mental and physical health outcomes, employment decisions (e.g., hiring and promotions), workplace outcomes (e.g., job satisfaction and performance), and among others;
Artificially Sourced Psychological Profile. Psychological profiles of individuals (e.g., world leaders, entertainers, etc.) can be generated via crowd sourcing or artificial intelligence, using publicly available information such as online media accounts, biographies, social networks, news outlets, etc.;
Psychological Profile Development Roadmap. As psychological profiles enable users to understand the underlining psychological makeup of others, they provide a roadmap for users to develop mindsets that emulate others. For example, if users wants to emulate an admirable person with “happiness” and “leadership” as mindset goals (i.e., the admirable person's psychological profile), then these mindset goals serve as a development roadmap whereby users may adopt these mindset goals for their own mindset training;
User Prompting. Users receive scheduled (e.g., daily, weekly, etc.) reminders and encouragement to engage with training. The computer system may be programmed to automatically send user prompts to user devices (e.g., utilizing chatbots or artificial intelligence). For example and without limitations, users may be reminded daily to curate mindset content or play the conditioning games;
Behavior Prompting. Users receive behavioral prompts that reinforce their mindset goals. The computer system may be programmed to automatically send behavioral prompts to user devices (e.g., utilizing chatbots or artificial intelligence). For example and without limitations, a user pursuing a happiness mindset goal may receive a prompt (e.g., “What behavior can you do today to make you happier?”) that encourage the user to perform the behavior. Similarly, the user may receive a prompt suggesting behaviors that reinforce their mindset goals (e.g. write a letter of appreciation to friends and family to induce happiness);
User Surveys. Users may be surveyed on a variety of topics. The computer system may be programmed to automatically send surveys to user devices (e.g., utilizing chatbots or artificial intelligence). For example, surveys could assess mindset training progress at regular intervals (e.g. “how happy are you today?”) that can serve as a metric to track progress towards mindset goals. Other topics such as voting behavior, product purchases, demographic, restaurant opinions, and among others, could be administered to users that can be analyzed with users' psychological profile for developing artificial intelligence and prediction systems;
Psychographic Data Mining. The database 107 will be become increasingly rich with usage overtime. For example and without limitations, the richness of data may include different domains of mindset training goals, user training engagement, social network interactions, demographics, behaviors, and a host of topics such as those gathered from surveys. Whereas demographic data tell who the users are (age, gender, sex, etc.), the data herein reflect psychographic data that tell why users think and behave in certain ways (i.e., mindset). Accordingly, the psychographic data may be mined for insights into developing artificial intelligence and prediction systems for cognition and behavior. For example and without limitations, insights gained from psychographic data mining may improve training efficacy by matching users to form social networks, suggesting mindset goals and content, providing behavioral prompts, motivating users (e.g., sending an encouraging message prompt “you've trained 5 days in a row, keep going and train now!”), recommending conditioning games users may like, selecting and managing game stimuli (e.g., presenting the most salient, recent, or most liked game stimuli), organizing optimal mindset training schedules, adjusting game difficulty levels, and among others. Psychographic data may also be utilized to predict a broad range of cognitions, behaviors, and outcomes, such as product purchases, branding preferences, voting behaviors, dating preferences, mental and physical health outcomes, employment decisions (e.g., hiring and promotions), workplace outcomes (e.g., job satisfaction and performance), and among others; and
User Engagement Incentives. User engagement can be enhanced by providing an incentive to curate mindset content or train mindset goals via conditioning games. Such incentive may comprise earning points to obtain a reward, points convertible to monetary value, points relating to progress and/or points for comparison between different users.

Accordingly, the scope should be determined not by the embodiments illustrated, but by the appended claims and their legal equivalents. Reference herein to “one embodiment” means that a particular feature, structure, or characteristic described in connection with the embodiment can be included in at least one embodiment of the invention. The appearances of the phrase “one embodiment” or “another embodiment” in various places in the specification are not necessarily all referring to the same embodiment, nor are separate or alternative embodiments necessarily mutually exclusive of other embodiments.

Although the invention has been set forth in terms of the exemplary embodiments described herein and illustrated in the attached documents, it is to be understood that such invention is purely illustrative and is not to be interpreted as limiting. Consequently, various alterations, modifications, and/or alternative embodiments and applications may be suggested to those skilled in the art after having read this disclosure. Accordingly, it is intended that the invention be interpreted as encompassing all alterations, modifications, or alternative embodiments and applications as fall within the true spirit and scope of this disclosure.

Although the present disclosure has been described in detail with reference to particular embodiments, implementations, arrangements and configurations, these exemplary embodiments, implementations, configurations and arrangements may be changed significantly without departing from the scope of the present disclosure. Moreover, although the exemplary embodiments have been illustrated with reference to particular elements and operations that facilitate the communication process, these elements, and operations may be replaced by any suitable architecture or process that achieves the intended functionality of the exemplary embodiments. Numerous other changes, substitutions, variations, alterations, and modifications may be ascertained to one skilled in the art and it is intended that the present disclosure encompass all such changes, substitutions, variations, alterations, and modifications falling within the scope of the appended claims.

It is understood that any specific order or hierarchy of steps in any disclosed process is an example of a sample approach. Based upon design preferences, it is understood that the specific order or hierarchy of steps in the processes may be rearranged while remaining within the scope of the present disclosure. The accompanying system and method claims present elements of the various steps in a sample order, and are not meant to be limited to the specific order or hierarchy presented.

1. A method for training a mindset of a user with gamification, the method being performed by a computing device, the method comprising:
specifying one or more mindset goals by the user;
curating mindset content by the user; and
conditioning the mindset of the user via one or more conditioning games.

2. The method of claim 1, wherein the mindset content is capable of representing and activating concepts related to the one or more mindset goals, and the mindset content may comprise a visual, an auditory, a gustatory, an olfactory, a tactile representation, or a combination thereof.

3. The method of claim 1, wherein the one or more mindset goals represent the unique mindset of the user.

4. The method of claim 1, wherein the mindset content is unique to the user.

5. The method of claim 1, wherein the mindset content simultaneously represents a conditioned stimulus and an unconditioned stimulus.

6. The method of claim 1, wherein the mindset content is transformed into one or more game stimuli by the computing device for use as inputs in one or more conditioning games, the conditioning games comprising one or more the stimuli.

7. The method of claim 1, wherein the one or more conditioning games comprise of dynamic combinations of one or more the conditioning games with one or more the game stimuli.

8. The method of claim 1, further comprising:
conditioning the mindset of the user, wherein the curating mindset content is directed to conditioning the user in the one or more mindset goals.

9. The method of claim 1, further comprising:
conditioning the mindset of the user, wherein the one or more conditioning games are directed to conditioning the user in the one or more mindset goals.

10. The method of claim 1, further comprising:
registering users as members of a social network;
matching and connecting the members of the social network, and permitting the members of the social network to search for matches and connect with other members of the social network;
accessing, by a member of the social network, the mindset content of other members of the social network;
presenting, to the accessing member, the mindset content of other members of the social network;
curating, by the accessing member, the mindset content of other members of the social network wherein the mindset content is associated with the accessing member's the one or more mindset goals; and
commenting, sharing, and liking the mindset content by the members of the social network.

11. A computer system for managing mindset training applications receiving data associated with users operating a plurality of devices in a communication network and delivering interventions to the users, the computer system includes application programs that are executed to cause the computer system to:
permit the users to specify and input one or more mindset goals;
permit the users to curate and upload mindset content; and
condition mindset of the users via one or more conditioning games.

12. The computer system of claim 11, wherein the mindset content are capable of representing and activating concepts related to the one or more mindset goals, and the mindset content may comprise a visual, an auditory, a gustatory, an olfactory, a tactile representation, or a combination thereof.

13. The computer system of claim 11, wherein the one or more mindset goals represent the unique mindset of the user.

14. The computer system of claim 11, wherein the application programs are further executed to cause the computer system to:
permit the users to curate and upload user-unique mindset content.

15. The computer system of claim 11, wherein the application programs are further executed to cause the computer system to:
permit the users to curate user-unique content that simultaneously represents a conditioned stimulus and an unconditioned stimulus.

16. The computer system of claim 11, wherein the application programs are further executed to cause the computer system to:
transform the content into one or more game stimuli for use as inputs in one or more conditioning games, the conditioning games comprising one or more the stimuli.

17. The computer system of claim 11, wherein the application programs are further executed to cause the computer system to:
provide a variety of the conditioning games by configuring dynamic combinations of one or more the conditioning games with one or more the game stimuli.

18. The computer system of claim 11, wherein the application programs are further executed to cause the computer system to:
condition the mindset of the users, wherein the curated mindset content is directed to conditioning users in the one or more mindset goals.

19. The computer system of claim 11, wherein the application programs are further executed to cause the computer system to:
condition the mindset of the users, wherein the one or more conditioning games are directed to conditioning the users in the one or more mindset goals.

20. The computer system of claim 11, wherein the application programs are further executed to cause the computer system to:
permit the users to register as members of the social network;
generate member matches and connections, and permit the members of the social network to search for matches and connect with other members of the social network;
permit the members of the social network to access mindset content of other members of the social network;
present to accessing members, the mindset content of other members of the social network;
permit to the accessing members to curate the mindset content of other members, wherein the mindset content of the other members is associated with the accessing members' one or more mindset goals; and
permit the members of the social network to commenting, share, and like the mindset content of other members of the social network.
